(19) United States
(12) Patent Application Publication
(10) Pub. No.: US 2011/0268653 A1
(43) Pub. Date: Nov. 3, 2011

(54) Compositions and Methods Related to Acid Stable Lipid Nanospheres

(75) Inventors: George R. Negrete, San Antonio, TX (US); Mathew P.D. Mahindaratne, San Antonio, TX (US); Adelph M. Mfuh, San Antonio, TX (US); Maritza V. Quintero, Salt Lake City, UT (US)

(73) Assignee: The Board of Regents of the University of Texas System, Austin, TX (US)

(21) Appl. No.: 13/087,952

(22) Filed: Apr. 15, 2011

Related U.S. Application Data
(60) Provisional application No. 61/325,111, filed on Apr. 16, 2010.

(51) Int. Cl. A61K 51/00; A61K 9/127; A61K 47/14; A61K 39/00; A61K 31/7088; A61K 47/36; A61K 49/00; A61K 39/395

(52) U.S. Cl. 424/1.11; 424/9.1; 424/450; 424/130.1; 424/184.1; 514/44 R; 514/777; 514/785

(57) ABSTRACT

The present invention relates generally to the fields of chemistry and biochemistry. More particularly, it concerns methods and compositions for the use of fatty asparagine, fatty cysteine, and fatty serine derivatives.

This application claims priority to U.S. Provisional Patent application Ser. No. 61/325,111 filed Apr. 16, 2010, which is incorporated herein by reference in its entirety.

This invention was made with government support under GM081194 awarded by National Institute of Health. The government has certain rights in the invention.

### BACKGROUND OF THE INVENTION

I. Field of the Invention

The present invention relates generally to the fields of chemistry and biochemistry. More particularly, it concerns methods and compositions for the use of fatty asparagine, fatty cysteine, and fatty serine derivatives.

II. Background

Liposomes are spherical bilayer vesicles that are under investigation as nanocapsules for transporting therapeutic (Torchilin, 2005; Gulati et al., 1998; Sharma and Sharma, 1997; Gabizon, 2006; Crommelin and Schreier, 1994) and non-therapeutic materials (Lasic and Barenholz, 1996; Stanzi, 1999; Handjani-Vila et al., 1993; Hayward and Smith, 1990; Strauss, 1989; Xia and Xu, 2005; Keller 2001; Picón et al., 1997; Kirby 1993; Kirby, 1991; Buttino et al., 2006). In medicinal applications, nanosphere encapsulation affords the potential for reduced toxicity (Torrado et al., 2008; Allen and Cullis, 2004; Chonn and Cullis 1995), improved bioavailability (Wang, 2005; Kshirsagar et al., 2005; Papahadjopoulos et al., 1991), and tissue-selective delivery (Sou et al., 2007; Vikbjerg et al. 2007; Rivest et al., 2007; Schiffelers et al., 2004; Kubo et al., 2003; Park, 2002; \check{S}entjurc et al., 1999; Phillips and Goins, 1995; Siegal et al., 1995). The increasing availability of liposomal medicines attests to the emerging therapeutic value of this modality (Michaleket al., 2005; Gregoriadis, 1988; Alving, 1987). An important challenge that remains to be addressed is the development of liposomes that are compatible with oral administration (De\check{g}im et al., 2006; Lee et al., 2004; Al-Meshal et al., 1998; Arién et al., 1993, Gregory et al., 1986). Such liposomes must survive the harsh gastrointestinal environments including severe stomach acidity (pH 1-2) before reaching the small intestines where ingested substances are disassembled and absorbed (Guyton and Hall, 2006; Camilleri et al., 1989). Liposomes have been fortified for enhanced stability with cholesterol (López-Pinto et al., 2005; McLean and Phillips, 1981; Kirby et al., 1980), triterpinoids (Valenti et al., 2001; Han et al., 1997; Nagumo et al., 1991), polyelectrolyte coatings (Mansy, 2009; Morigaki and Walde, 2007; Sakaguchi et al., 2008; Osanai and Nakamura, 2000; Müller et al., 2005; Gillies and Fréchet, 2004; Dong and Rogers, 1992), and lipid cross-linking (Lee et al., 2007; Regen, 1987; Ng et al., 2001; Lawson et al., 2005; Halter et al., 2004; Lawson et al., 2003; Werle and Takeuchi, 2009; Carafa et al., 2006; Kulkarni et al., 1995; Dong and Rogers, 1993; Cohen et al., 1991; Kibat et al., 1990).

There remains a need for liposomes that are stable under acidic conditions.

### SUMMARY OF THE INVENTION

Embodiments are directed to stabilized lipid compositions and the methods for using the same. In particular, compositions of the invention are stable in an acidic environment for extended periods of time. Embodiments include methods of exploiting the acid stability of the lipid particles to deliver therapeutic and diagnostic components through the stomach and to the intestines. In certain aspects the lipid particles can be used in compositions and methods that require lipid particle stability at low pH.

Certain embodiments are directed to methods of administering a therapeutic or diagnostic agent to a subject comprising orally administering a lipidic particle comprising at least 5, 10, 15, 20, 40, 50, or 60 mol %, including all values and ranges there between, of asparagine-derived lipid analogs (ALA), cysteine-derived lipid analogs (CLA), or serine-derived lipid analogs (SLA), and a therapeutic or diagnostic agent to a subject. The lipidic particle can further comprise 40, 50, 60, 70, 80, 90, or 95 mol %, including all values and ranges there between, of a phospholipid. In certain aspects the phospholipid is phosphatidylcholine. In a further aspect, the phosphatidylcholine is distearoylphosphatidylcholine (DSPC). The lipidic particle can further comprise 0.01 0.05, 0.5, 1, 5, 10, 15 to 20 mol %, including all values and ranges there between, of a stabilizing agent. In certain aspects, the stabilizing agent is cholesterol, cholesterol esters, cholestanol, glucoronic acid derivatives, polysaccharide, saturated fatty acids, unsaturated fatty acids, and/or polyethylene glycol. In a further aspect the lipidic particle is a liposome. The therapeutic or diagnostic agent can be an antigen, an antibiotic, a peptide, a pharmaceutical, a nucleic acid, a detectable agent, and/or an antibody. In certain aspects the detectable agent is a radiographic contrast agent.

Further embodiments are directed to methods of delivering an antigen to the small intestines comprising orally administering an antigen encapsulated in a lipidic particle comprising at least 5, 10, 15, 20, 40, or 60 mol % ALA, CLA, or SLA, and an encapsulated acid labile agent to a subject.

Certain embodiments are directed to an acid stable lipid particle composition comprising: (a) at least 5, 10, 15, 20, 40, or 60 mol %, including all values and ranges there between, of a lipopeptide derivative selected from an asparagine, cysteine, or serine lipid derivative having the formula ALA_{R1,R2}, CLA_{R1,R2}, or SLA_{R1,R2} wherein in R1 and R2 are independently an alkyl chain of at least 10, 11, 12, 13, 14, 15, 16, 17, 18, 19, or 20 carbons; (b) at least 40, 60, 80, 90, 95 mol % amphiphilic lipid; and (c) a therapeutic or diagnostic agent; wherein the lipid particle is stable for at least 1, 2, 3, 4, 5, 6, 7, 8, 9, 10, 11, 12, 13, 14, 15, 16, 17, 18, 19, 20, 21, 22, 23, 24, 48, 72, 96, 120 or more hours, including all values and ranges there between, at a pH of 0.5, 1, 2, 3 to 2, 3, 4, 5, 6, including all values and ranges there between, at room temperature. In certain aspects R1 is 9, 10, 11, or 12; and R2 is 15, 16, 17, 18, 19, or 20. In a certain aspect R1 is 11 and R2 is 17. In a further aspect the lipidic particles are 1, 10, 20, 40, 50, 100 μm to 100, 150, 200, 250, 300 μm or nm in diameter, including all ranges and values there between. The size distribution can be uniform and with +/−5, 10, 20, 30, 40, 50 μm or nm. The particle can further comprising a targeting moiety. In certain aspects the particle can further comprising a detectable label.

Certain embodiments are directed to methods of modulating lipidic particle size by adjusting the ALA, CLA, and/or SLA content. An increase in ALA. CLA, and/or SLA results in a decrease in lipidic particle size.

Further embodiments are directed to methods of modulating acid stability of a lipid particle by adjusting the length of the alkyl chains of a ALA, CLA, or SLA component of the lipid particle, wherein the shorter the alkyl chain the less stable the lipid particle.

Still further embodiments are directed to methods of preparing a lipid particle having a size range of 5, 10, 20, 30, 40, 50, 60, 70, 80, 90, 100 μm to 100. 125, 150, 200, 250, 300 μm, including all values and ranges there between, without extruding the lipidic mixture comprising: (a) combining an ALA, CLA, or SLA, and an amphiphilic lipid, wherein the ALA, CLA, or SLA component is 5, 10, 20, 40, or 60 mol % of the combination and the amphiphilic component is 40, 50, 60, 70, 80, 90, 95 mol % of the combination; (b) preparing a thin film of the combination; (c) hydrating the thin film forming self-assembled lipid particles comprising at least 5 mol % ALA, CLA, or SLA.

A “lipidic particle” refers to a particle having a membrane structure in which amphipathic lipid molecules are arranged with their polar groups oriented to an aqueous phase. Examples of the lipid membrane structure include configurations such as a liposome, multi-lamellar vesicle (MLV), and a micelle structure.

A “liposome” refers to a closed nanosphere, which is formed by forming a bilayer membrane of a phospholipid molecule with the hydrophobic moiety positioned inside and the hydrophilic moiety positioned outside, in water and closing the ends of the bilayer membrane. Examples of liposome include a nanosphere having a single layer formed of a phospholipid bilayer membrane and a nanosphere having a multiple layer formed of a plurality of phospholipid bilayers. Since a liposome has such a structure, an aqueous solution is present both inside and outside of the liposome and the lipid bilayer serves as the boundary.

A “micelle” refers to an aggregate of amphipathic molecules. The micelle has a form in which a lipophilic moiety of this amphipathic molecules is positioned toward the center of the micelle and a hydrophilic moiety is positioned toward the outside thereof, in an aqueous medium. A center of a sphere is lipophilic and a peripheral portion is hydrophilic in such a micelle. Examples of a micelle structure include spherical, laminar, columnar, ellipsoidal, microsomal and lamellar structures, and a liquid crystal.

As used herein, the term “antigen” is a molecule capable of being bound by an antibody or T-cell receptor. An antigen is additionally capable of inducing a humoral immune response and/or cellular immune response leading to the production of B- and/or T-lymphocytes. The structural aspect of an antigen that gives rise to a biological response is referred to herein as an “antigenic determinant.” B-lymphocytes respond to foreign antigenic determinants via antibody production, whereas T-lymphocytes are the mediator of cellular immunity. Thus, antigenic determinants or epitopes are those parts of an antigen that are recognized by antibodies, or in the context of an MHC, by T-cell receptors. An antigenic determinant need not be a contiguous sequence or segment of protein and may include various sequences that are not immediately adjacent to one another.

Other embodiments of the invention are discussed throughout this application. Any embodiment discussed with respect to one aspect of the invention applies to other aspects of the invention as well and vice versa. The embodiments in the Example section are understood to be embodiments of the invention that are applicable to all aspects of the invention.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed herein can be implemented with respect to any method or composition of the invention, and vice versa. Furthermore, compositions and kits of the invention can be used to achieve methods of the invention.

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.” It is also contemplated that anything listed using the term “or” may also be specifically excluded.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1D. The high resolution optical microscopy (HROM) images of selective ALA_{11,17}/DSPC multilamellar vesicles (MLVs) in different compositions at pH 7.4 (PBS buffer): (a) DSPC MLVs; (b) 10 mol % ALA_{11,17}/DSPC MLVs; (c) 25 mol % ALA_{11,17}/DSPC MLVs; and (d) 50 mol % ALA_{11,17}/DSPC MLVs.

FIGS. 2A-2F. The HROM images of 10 mol % ALA_{n,m}/DSPC MLVs at pH 7.4 (PBS buffer): (a) ALA_{5,5}; (b) ALA_{5,11}; (c) ALA_{11,5}; (d) ALA_{11,11}; (^{e}) ALA_{17,11}; and (f) ALA_{17,17}.

FIGS. 3A-3B. Scanning electron micrographs of vortexed lipid self-assemblies after negative staining with ammonium molybdate at pH 7.4 (PBS buffer): (a) DSPC MLVs and (b) 10 mol % ALA_{11,17}/DSPC MLVs.

FIGS. 4A-4D. Dynamic Light Scattering Analysis showing the radii and size distribution of liposome suspension at different pH: (a) DSPC at pH 7.4 (PBS); (b) 10 mol % ALA_{11,17}/DSPC at pH 7.4 (PBS); (c) DSPC at pH 1.9 (PBS/HCl); and (d) 10 mol % ALA_{11,17}/DSPC at pH 1.9 (PBS/HCl).

FIGS. 5A-5D. Scanning electron micrographs of negatively stained liposome samples prepared by three-time extrusions through each of polycarbonate filters with pore sizes of 2.0, 1.0, 0.40, 0.20, and 0.10 μm at pH 7.4 (PBS buffer). (a) DSPC formulation; (b) 10 mol % ALA_{11,17}/DSPC formulation; (c) 10 mol % ALA_{11,17}/DSPC formulation of 5b extruded extra 8 times through 0.1 μm filter; and (d) Same sample of 5c at different magnification.

FIG. 6. Normalized absorbances of liposomal solutions of 0, 5, 10, 15, 25, and 50 mol % ALA_{11,17}/DSPC in PBS at 400 nm in various pH (see the insertion for the color code of corresponding plots). The pH was changed by the additions of appropriate aliquots of 1% HCl (v/v).

FIGS. 7A-7B. Scanning electron microscopy images of negatively stained (a) DSPC and (b) 10% ALA_{11,17}/DSPC liposomes at pH 1.9 (PBS/HCl). Liposomes were negatively stained by ammonium molybdate.

FIGS. 8A-8B. Change of normalized absorbances of the liposomal solutions of DSPC, 5 mol % ALA_{11,17}/DSPC, and 10 mol % ALA_{11,17}/DSPC in PBS at 400 nm with time: (A) at pH 7.4 (PBS buffer) and (B) at pH 1.9 (PBS/HCl). (a) DSPC liposomes at pH 7.4; (b) 5 mol % ALA_{11,17}/DSPC at pH 7.4; (c) 10 mol % ALA_{11,17}/DSPC liposomes at pH 7.4; (d) DSPC liposomes at pH 1.9; (e) 5 mol % ALA_{11,17}/DSPC at pH 1.9; and (f) 10 mol % ALA_{11,17}/DSPC liposomes at pH 1.9.

FIG. 9. Provides an illustration of a scheme for preparation of 1,3-cis-substituted tetahydropyrimdinones from L-aspargine and a schematic of conformers of ALA_{5,11} n solution.

### DETAILED DESCRIPTION OF THE INVENTION

Amino acid-derived lipid analogues bearing a tetrahydropyrimidinone head group and two fatty chains (e.g., XLA_{n,m} where X is asparagine (A), serine (S), or cysteine (C) and n and m indicate the lengths of linear alkyl chains, also designated R1 and R2 respectively) can be synthesized in high yield and purity. The lipids are characterized by spectroscopic and other physical methods. Multilamellar vesicles (MLVs) can be formed upon hydration of mixtures of XLA_{n,m} and phospholipids. The MLVs can be processed into unilamellar nanospheres via extrusion and characterized by dynamic light scattering (DLS) and scanning electron microscopy (SEM). The comparative acid stabilities of liposome formulations with and without XLA_{n,m} can be interrogated by turbidity, DLS, and SEM. Turbidity studies indicated that 10 mol % or higher ALA_{11,17}/DSPC liposome formulations persisted at pH 1.9, which is unprecedented for phosphatidylcholine liposomes. Vesicles prepared with smaller proportions of ALA_{11,17} degraded below pH 4.2. These findings were confirmed by SEM experiments. The turbidity studies also suggested that liposomes with greater proportions of ALA_{11,17} (15, 25, and 50 mol %) aggregated near pH 3 and reverted to isolated nanospheres upon further acidification.

### I. LIPIDIC PARTICLES

In certain embodiments, the lipidic particles are microparticles or nanoparticles that include at least one lipid component forming a condensed lipid phase. Typically, a lipidic particle has preponderance of lipids in its composition. The exemplary condensed lipid phases are solid amorphous or true crystalline phases; isomorphic liquid phases (droplets); and various hydrated mesomorphic oriented lipid phases such as liquid crystalline and pseudocrystalline bilayer phases (L-alpha, L-beta, P-beta, Lc), interdigitated bilayer phases, and nonlamellar phases (inverted hexagonal H-I, H-II, cubic Pn3m) (see The Structure of Biological Membranes, ed. by P. Yeagle, CRC Press, Bora Raton, Fla., 1991, in particular ch. 1-5, incorporated herein by reference). Lipidic particles include, but are not limited to a liposome. Methods of making and using these types of lipidic particles, as well as attachment of affinity moieties, e.g., antibodies, to them are known in the art (see, e.g., U.S. Pat. Nos. 5,077,057; 5,100,591; 5,616,334; 6,406,713 (drug-lipid complexes); U.S. Pat. Nos. 5,576,016; 6,248,363; Bondi et al., 2003; Pedersen et al., 2006 (solid lipid particles); U.S. Pat. Nos. 5,534,502; 6,720,001; Shiokawa et al., 2005 (microemulsions); U.S. Pat. No. 6,071,533 (lipid-nucleic acid complexes)).

A liposome is generally defined as a particle comprising one or more lipid bilayers enclosing an interior, typically an aqueous interior. Thus, a liposome is often a vesicle formed by a bilayer lipid membrane. There are many methods for the preparation of liposomes. Some of them are used to prepare small vesicles (d<0.05 micrometer), some for larger vesicles (d>0.05 micrometer). Some are used to prepare multilamellar vesicles, some for unilamellar ones. Methods for liposome preparation are exhaustively described in several review articles such as Szoka and Papahadjopoulos (1980); Deamer and Uster (1983), and the like.

In various embodiments, liposomes of the invention are composed of vesicle-forming lipids, generally including amphipathic lipids having both hydrophobic tail groups and polar head groups. A characteristic of a vesicle-forming lipid is its ability to either (a) form spontaneously into bilayer vesicles in water, as exemplified by the phospholipids, or (b) be stably incorporated into lipid bilayers, by having the hydrophobic portion in contact with the interior, hydrophobic region of the bilayer membrane, and the polar head group oriented toward the exterior, polar surface of the membrane. A vesicle-forming lipid for use in the present invention is any conventional lipid possessing one of the characteristics described above.

In certain embodiments the vesicle-foil ling lipids of this type are preferably those having two hydrocarbon tails or chains, typically acyl groups, and a polar head group. Included in this class are the phospholipids, such as phosphatidylcholine (PC), phosphatidylethanolamine (PE), phosphatidic acid (PA), phosphatidylglycerol (PG), and phosphatidylinositol (PI), where the two hydrocarbon chains are typically between about 14-22 carbon atoms in length, and have varying degrees of unsaturation. In certain embodiments preferred phospholipids include PE and PC. One illustrative PC is hydrogenated soy phosphatidylcholine (HSPC). Single chain lipids, such as sphingomyelin (SM), and the like can also be used.

The above-described lipids and phospholipids whose acyl chains have a variety of degrees of saturation can be obtained commercially, or prepared according to published methods. Other lipids that can be included in certain embodiments are sphingolipids and glycolipids. The term “sphingolipid” as used herein encompasses lipids having two hydrocarbon chains, one of which is the hydrocarbon chain of sphingosine. The term “glycolipids” refers to shingolipids comprising also one or more sugar residues.

Lipids for use in the lipidic particles of the present invention can include relatively “fluid” lipids, meaning that the lipid phase has a relatively low lipid melting temperature, e.g., at or below room temperature, or alternately, relatively “rigid” lipids, meaning that the lipid has a relatively high melting point, e.g., at temperatures up to 50° C. As a general rule, the more rigid, i.e., saturated lipids, contribute to greater membrane rigidity in the lipid bilayer structure, and thus to more stable drug retention after active drug loading. In certain embodiments preferred lipids of this type are those having phase transition temperatures above about 37° C.

In various embodiments the liposomes may additionally include lipids that can stabilize a vesicle or liposome composed predominantly of phospholipids. An illustrative lipid of this group is cholesterol at levels between 1 to 45 mole percent.

In certain embodiments liposomes used in the invention contain between 30-75 percent phospholipids, e.g., phosphatidylcholine (PC), and 5-45 percent XLA. One illustrative liposome formulation contains 60 mole percent phosphatidylcholine and 40 mole percent XLA.

In certain aspects, the liposomes may include a surface coating of a hydrophilic polymer chain. “Surface-coating” refers to the coating of any hydrophilic polymer on the surface of liposomes. The hydrophilic polymer is included in the liposome by including in the liposome composition one or more vesicle-forming lipids derivatized with a hydrophilic polymer chain.

In certain embodiments a hydrophilic polymer for use in coupling to a vesicle forming lipid is polyethyleneglycol (PEG), preferably as a PEG chain having a molecular weight between 1,000-10,000 Daltons, more preferably between 1,000-5,000 Daltons, most preferably between 2,000-5,000 Daltons. Methoxy or ethoxy-capped analogues of PEG are also useful hydrophilic polymers, commercially available in a variety of polymer sizes, e.g., 120-20,000 Daltons.

Other hydrophilic polymers that can be suitable include, but are not limited to polylactic acid, polyglycolic acid, polyvinylpyrrolidone, polymethyloxazoline, polyethyloxazoline, polyhydroxypropyl methacrylamide, polymethacrylamide, polydimethylacrylamide, and derivatized celluloses, such as hydroxymethylcellulose or hydroxyethylcellulose.

Preparation of lipid-polymer conjugates containing these polymers attached to a suitable lipid, such as PE, has been described, for example in U.S. Pat. No. 5,395,619, which is expressly incorporated herein by reference, and by Zalipsky in STEALTH LIPOSOMES (1995). In certain embodiments, typically, between about 1-20 mole percent of the polymer-derivatized lipid is included in the liposome-forming components during liposome formation. Polymer-derivatized lipids suitable for practicing the invention are also commercially available (e.g. SUNBRITE®, NOF Corporation, Japan).

The liposomes may be prepared by a variety of techniques, such as those detailed in Szoka and Papahadjopoulos (1980), and a specific example of liposomes prepared in support of the present invention is set forth in the Examples section. In certain embodiments the liposomes are multilamellar vesicles (MLVs), which can be formed by lipid-film hydration techniques. In this procedure, a mixture of liposome-forming lipids and XLA are dissolved in a suitable organic solvent which is evaporated in a vessel to form a dried thin film. The film is then covered by an aqueous medium to form MLVs, typically with sizes between about 0.1 to 10 microns. Illustrative methods of preparing derivatized lipids and of forming polymer-coated liposomes have been described in U.S. Pat. Nos. 5,013,556, 5,631,018, and 5,395,619, which are incorporated herein by reference.

After liposome formation, the vesicles may be sized to achieve a size distribution of liposomes within a selected range, according to known methods. In certain embodiments the liposomes are uniformly sized to a selected size range between 0.04 to 0.25 microns. Small unilamellar vesicles (SUVs), typically in the 0.04 to 0.08 micron range, can be prepared by extensive sonication or homogenization of the liposomes. Homogeneously sized liposomes having sizes in a selected range between about 0.08 to 0.4 microns can be produced, e.g., with or without extrusion through polycarbonate membranes or other defined pore size membranes having selected uniform pore sizes ranging from 0.03 to 0.5 microns, typically, 0.05, 0.08, 0.1, or 0.2 microns. The pore size of the membrane corresponds roughly to the largest size of liposomes produced by extrusion through that membrane, particularly where the preparation is extruded two or more times through the same membrane. The sizing is typically carried out in the original lipid-hydrating buffer, so that the liposome interior spaces retain this medium throughout the initial liposome processing steps.

In one illustrative approach a mixture of liposome-forming lipids is dissolved in a suitable organic solvent and evaporated in a vessel to form a thin film. The film is then covered with an aqueous medium containing the solute species that will form the aqueous phase in the liposome interior spaces in the final liposome preparation. The lipid film hydrates to form multi-lamellar vesicles (MLVs), typically with heterogeneous sizes between about 0.1 to 10 microns. The liposome are then sized, as described above, to a uniform selected size range.

While the foregoing discussion pertains to the formation of liposomes, similar lipids and lipid compositions can be used to form other lipidic microparticles or nanoparticles such as a solid lipid particle, a microemulsion, and the like.

Methods of functionalizing lipids and liposomes with affinity moieties such as antibodies are well known to those of skill in the art (see, e.g., DE 3,218,121; Epstein et al. (1985); Hwang et al. (1980); EP 52,322; EP 36,676; EP 88,046; EP 143,949; EP 142,641; Japanese patent application 83-118008; U.S. Pat. Nos. 4,485,045 and 4,544,545; and EP 102,324, all of which are incorporated herein by reference).

Microparticle and especially nanoparticle-based drug delivery systems have considerable potential for treatment of various pathologies. Technological advantages of polymeric microparticles or nanoparticles used as drug carriers is high stability, high carrier capacity, feasibility of incorporation of both hydrophilic and hydrophobic substances, and feasibility of variable routes of administration, including oral application and inhalation.

The particles described herein are typically micron or submicron (<1 micrometer) particles. In certain embodiments, the drug can be covalently attached to the surface or located with in the particle or located with in the lipid environment or combinations thereof.

A. Therapeutic Agents

In one embodiment, a therapeutic agent is introduced into the lipid particle. By “therapeutic agent” or “drug moiety” or “therapeutically active agent” herein is meant that an agent is capable of affecting a therapeutic effect, i.e. it alters a biological function of a physiological target substance. By “causing a therapeutic effect” or “therapeutically effective” or grammatical equivalents herein is meant that the agent alters the biological function of its intended physiological target in a manner sufficient to cause a therapeutic and phenotypic effect. By “alters” or “modulates the biological function” herein is meant that the physiological target undergoes a change in either the quality or quantity of its biological activity; this includes increases or decreases in activity. Thus, therapeutically active agents include a wide variety of drugs, including antagonists, for example enzyme inhibitors, and agonists, for example a transcription factor which results in an increase in the expression of a desirable gene product (although as will be appreciated by those in the art, antagonistic transcription factors may also be used), are all included.

In addition, a “therapeutic agent” includes those agents capable of direct toxicity and/or capable of inducing toxicity towards healthy and/or unhealthy cells in the body. Also, the therapeutic agent may be capable of inducing and/or priming the immune system against potential pathogens. A number of mechanisms are possible including without limitation, (i) a radioisotope linked to a protein as is the case with a radiolabled protein, (ii) an antibody linked to an enzyme that metabolizes a substance, such as a prodrug, thus rendering it active in vivo, (iii) an antibody linked to a small molecule therapeutic agent, (iv) a radioisotope, (v) a carbohydrate, (vi) a lipid, (vii) a thermal ablation agent, (viii) a photosensitizing agent, (ix) a vaccine agent and the like.

1. Small Molecules and Drugs

In one aspect, a lipid particle described herein can include therapeutic agents such as small molecules or drugs, for example a chemotherapeutic such as doxorubicin. In one embodiment, the particle can include an anti-cancer drug. In certain aspects the small molecule or drug can target a protein. The target protein can be an enzyme. As will be appreciated by those skilled in the art, the possible enzyme target substances are quite broad. Suitable classes of enzymes include, but are not limited to, hydrolases such as proteases, carbohydrases, lipases and nucleases; isomerases such as racemases, epimerases, tautomerases, or mutases; transferases, kinases and phophatases. Enzymes associated with the generation or maintenance of arterioschlerotic plaques and lesions within the circulatory system, inflammation, wounds, immune response, tumors, apoptosis, exocytosis, etc. may all be treated using the present invention. Enzymes such as lactase, maltase, sucrase or invertase, cellulase, alpha-amylase, aldolases, glycogen phosphorylase, kinases such as hexokinase, proteases such as serine, cysteine, aspartyl and metalloproteases may also be detected, including, but not limited to, trypsin, chymotrypsin, and other therapeutically relevant serine proteases such as tPA and the other proteases of the thrombolytic cascade; cysteine proteases including: the cathepsins, including cathepsin B, L, S, H, J, N and O; and calpain; and caspases, such as caspase-3, -5, -8 and other caspases of the apoptotic pathway, such as interleukin-converting enzyme (ICE). As will be appreciated in the art, this list is not meant to be limiting.

In another embodiment, the therapeutically active compound is a drug used to treat cancer. Suitable cancer drugs include, but are not limited to, antineoplastic drugs, including alkylating agents such as alkyl sulfonates (busulfan, improsulfan, piposulfan); aziridines (benzodepa, carboquone, meturedepa, uredepa); ethylenimines and methylmelamines (altretamine, triethylenemelamine, triethylenephosphoramide, triethylenethiophosphoramide, trimethylolmelamine); nitrogen mustards (chlorambucil, chlornaphazine, cyclophosphamide, estramustine, ifosfamide, mechlorethamine, mechlorethamine oxide hydrochloride, melphalan, novembichin, phenesterine, prednimustine, trofosfamide, uracil mustard); nitrosoureas (carmustine, chlorozotocin, fotenmustine, lomustine, nimustine, ranimustine); dacarbazine, mannomustine, mitobranitol, mitolactol; pipobroman; doxorubicin, carboplatin, oxaliplatin, and cisplatin, (including derivatives).

In some embodiments, the therapeutically active compound is an antiviral or antibacterial drug, including aclacinomycins, actinomycin, anthramycin, azaserine, bleomycins, cuctinomycin, carubicin, carzinophilin, chromomycins, ductinomycin, daunorubicin, 6-diazo-5-oxn-I-norieucine, duxorubicin, epirubicin, mitomycins, mycophenolic acid, nogalumycin, olivomycins, peplomycin, plicamycin, porfiromycin, puromycin, streptonigrin, streptozocin, tubercidin, ubenimex, zinostatin, zorubicin; aminoglycosides and polyene and macrolide antibiotics.

In other embodiments, the therapeutically active compound is a radio-sensitizer drug, which sensitizes cells to radiation. In one embodiment, the cells sensitized are tumor cells. These drugs may be used in conjunction with radiation therapy for cancer treatment. Radiosensitizer drugs include without limitation halogenated pyrimidines such as bromodeoxyuridine and 5-iododeoxyuridine (IUdR), caffeine, and hypoxic cell sensitizers such as isometronidazole.

In another embodiment, the therapeutic agent is a radioprotectant or radioprotector, which protects normal cells, such as non-tumor cells from any damage caused by radiation therapy of tumor cells. Examples of radioprotectants include without limitation amifostine (Ethyol®). In some embodiments, the therapeutically active compound is an anti-inflammatory drug (either steroidal or non-steroidal). In one embodiment, the therapeutically active compound is involved in angiogenesis. Suitable moieties include, but are not limited to, endostatin, angiostatin, interferons, platelet factor 4 (PF4), thrombospondin, transforming growth factor beta, tissue inhibitors of metalloproteinase-1, -2 and -3 (TIMP-1, -2 and -3), TNP-470, Marimastat, Neovastat, BMS-275291, COL-3, AG3340, Thalidomide, Squalamine, Combrestastatin, SU5416, SU6668, IFN-.alpha., EMD121974, CAI, IL-12 and IM862.

An antimicrobial is a substance that kills or inhibits the growth of microorganisms such as bacteria, fungi, or protozoans. Antimicrobial drugs either kill microbes (microbicidal) or prevent the growth of microbes (microbistatic). Disinfectants are antimicrobial substances used on non-living objects. Antibiotics are commonly classified based on their mechanism of action, chemical structure or spectrum of activity. Most antibiotics target bacterial functions or growth processes. Antibiotics that target the bacterial cell wall (penicillins, cephalosporins), or cell membrane (polymixins), or interfere with essential bacterial enzymes (quinolones, sulfonamides) are usually bactericidal in nature. Those that target protein synthesis, such as the aminoglycosides, macrolides, and tetracyclines, are usually bacteriostatic. Further categorization is based on their target specificity: “narrow-spectrum” antibiotics target particular types of bacteria, such as Gram-negative or Gram-positive bacteria, while broad-spectrum antibiotics affect a wide range of bacteria. Three other classes of antibiotics include cyclic lipopeptides (daptomycin), glycylcyclines (tigecycline), and oxazolidinones (linezolid). Tigecycline is a broad-spectrum antibiotic, while the two others are used for Gram-positive infections. These developments show promise as a means to counteract the bacterial resistance to existing antibiotics.

2. Nucleic Acids

In one embodiment, the therapeutically active agent is a nucleic acid, for example nucleic acids used for gene therapy or antisense therapy. By “nucleic acid” or “oligonucleotide” or grammatical equivalents herein means at least two nucleotides covalently linked together. A nucleic acid of the present invention will generally contain phosphodiester bonds, although in some cases, for example when therapeutic antisense molecules may have alternate backbones, comprising, for example, phosphoramide (Beaucage et al., 1993 and references therein; Letsinger, 1970; Sprinzl et al., 1977; Letsinger et al., 1986; Sawai et al., 1984, Letsinger et al., 1988; and Pauwels et al., 1968), phosphorothioate (Mag et al., 1991; and U.S. Pat. No. 5,644,048), phosphorodithioate (Briu et al., 1989, O-methylphosphoroamidite linkages (see Eckstein, Oligonucleotides and Analogues: A Practical Approach, Oxford University Press), and peptide nucleic acid backbones and linkages (see Egholm, 1992; Meier et al., 1992; Nielsen, 1993; Carlsson et al., 1996, all of which are incorporated by reference).

In one embodiment, the nucleic acids suitable as agents are short interfering nucleic acid (siNA) molecules that act by invoking RNA interference. RNA interference mechanisms recognize RNA as “foreign” due to its existence in a double-stranded form. This results in the degradation of the double-stranded RNA, along with single-stranded RNA having the same sequence. Short interfering RNAs, or “siRNAs”, are an intermediate in the RNAi process in which the long double-stranded RNA has been cut up into short (−21 nucleotides) double-stranded RNA. The siRNA stimulates the cellular machinery to cut up other single-stranded RNA having the same sequence as the siRNA.

As will be appreciated by those in the art, all of these nucleic acid analogs may find use in the present invention. In addition, mixtures of naturally occurring nucleic acids and analogs can be made. Alternatively, mixtures of different nucleic acid analogs, and mixtures of naturally occurring nucleic acids and analogs may be made.

The nucleic acids may be single stranded or double stranded, as specified, or contain portions of both double stranded or single stranded sequence. The nucleic acid may be DNA, both genomic and cDNA, RNA or a hybrid, depending on its ultimate use, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc.

3. Proteins

In one aspect, the therapeutically active agent is a protein. By “proteins” or grammatical equivalents herein is meant proteins, oligopeptides and peptides, derivatives and analogs, including proteins containing non-naturally occurring amino acids and amino acid analogs, and peptidomimetic structures. The side chains may be in either the (R) or the (S) configuration. In one embodiment, the amino acids are in the (S) or L-configuration.

In another embodiment, the protein is an antibody. The term “antibody” includes monoclonal antibodies, polyclonal antibodies, and antibody fragments thereof. Specific antibody fragments include, but are not limited to, (i) the Fab fragment consisting of VL, VH, CL and CH1 domains, (ii) the Fd fragment consisting of the VH and CH1 domains, (iii) the Fv fragment consisting of the VL and VH domains of a single antibody; (iv) the dAb fragment (Ward et al., 1989) which consists of a single variable, (v) isolated CDR regions, (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., 1988; Huston et al., 1988), (viii) bispecific single chain Fv dimers (PCT/US92/09965) and (ix) “diabodies” or “triabodies”, multivalent or multispecific fragments constructed by gene fusion (Tomlinson et al., 2000; WO94/13804; Holliger et al., 1993). In one embodiment, the protein included is a monoclonal antibody.

A peptide can be any medically or diagnostically useful peptide or protein of small to medium size (i.e. up to about 15 kD, 30 kD, 40 kD, 50 kD, 60 kD, 70 kD, 80 kD, 90 kD, 100 kD, for example). The mechanisms of improved polypeptide absorption are described in U.S. Pat. No. 5,661,130 which is hereby incorporated by reference in its entirety. Compositions described herein can be mixed with all such peptides. Examples of polypeptides include vasopressin, vasopressin polypeptide analogs, desmopressin, glucagon, corticotropin (ACTH), gonadotropin, calcitonin, C-peptide of insulin, parathyroid hormone (PTH), growth hormone (HG), human growth hormone (hGH), growth hormone releasing hormone (GHRH), oxytocin, corticotropin releasing hormone (CRH), somatostatin or somatostatin polypeptide analogs, gonadotropin agonist or gonadotrophin agonist polypeptide analogs, human atrial natriuretic peptide (ANP), human thyroxine releasing hormone (TRH), follicle stimulating hormone (FSH), prolactin, insulin, insulin like growth factor-I (IGF-I) somatomedin-C (SM-C), calcitonin, leptin and the leptin derived short peptide OB-3, melatonin, GLP-1 or Glucagon-like peptide-1, GiP, neuropeptide pituitary adenylate cyclase, GM-1 ganglioside, nerve growth factor (NGF), nafarelin, D-tryp6)-LHRH, FGF, VEGF antagonists, leuprolide, interferon (e.g., alpha, beta, gamma) low molecular weight heparin, PYY, LHRH antagonists, Keratinocyte Growth Factor (KGF), Glial-Derived Neurotrophic Factor (GDNF), ghrelin, and ghrelin antagonists. Further, in some aspects, the peptide or protein is selected from a growth factor, interleukin, polypeptide vaccine, enzyme, endorphin, glycoprotein, lipoprotein, or a polypeptide involved in the blood coagulation cascade.

4. Carbohydrate and Lipid Therapeutic Agents

In one embodiment, the therapeutic agent is a carbohydrate. By “carbohydrate” it is meant a compound with the general formula Cx(H₂O)y. Monosaccharides, disaccharides, and oligo- or polysaccharides are all included within the definition and comprise polymers of various sugar molecules linked via glycosidic linkages. Suitable carbohydrates (particularly in the case of targeting moieties, described below) are those that comprise all or part of the carbohydrate component of glycosylated proteins, including monomers and oligomers of galactose, mannose, fucose, galactosamine, (particularly N-acetylglucosamine), glucosamine, glucose and sialic acid, and in particular the glycosylation component that allows binding to certain receptors such as cell surface receptors. Other carbohydrates comprise monomers and polymers of glucose, ribose, lactose, raffinose, fructose, and other biologically significant carbohydrates.

5. Inorganic Material Agents

In a one embodiment, the present invention provides a range of inorganic materials that can be included in the particles described herein, many of which have biomedical applications. The inorganic materials include without limitation: Fe₂O₃, Fe₃O₄, Mn₂O₃, Co₃O₄, CO₂O₃, TiO₂-x(OH)x, Eu₂O₃, ZnSe, ZnS, and metallic particles such as Pt, Au, FePt and CoPt (Allen et al., 2002; Allen et al., 2003; Douglas, 1996; Douglas et al. 1995; Douglas et al. 1995). These inorganic material agents may be used in various applications.

6. Agents for Photodynamic Therapy (PDT)

In one other aspect, the particles may be used in photodynamic therapy (PDT). PDT is a therapeutic treatment that utilizes a drug, usually a photosensitizer or photosensitizing agent and a particular type of light. (Dougherty et al., 1998). Upon exposure to a specific wavelength of light, certain photosensitizers produce a form of oxygen that is cytotoxic to cells in the area of treatment. A given photosensitizer is activated by light of a particular wavelength, which determines how far the light can travel through tissue. Different photosensitizers are therefore suitable for the application of PDT in different areas of the body. PDT is typically performed by administering a photosensitizer to a patient in need followed by exposure of the treated area to light capable of exciting the photosensitizer. In the presence of molecular oxygen, an energy transfer occurs resulting in production of the highly cytotoxic singlet oxygen (1O₂), which is a very aggressive chemical species capable of reacting with biomolecules in its vicinity. PDT is known to be effective as a cancer treatment in multiple ways, including without limitation killing tumor cells directly, damaging blood vessels in a tumor and activation of the immune system to destroy the tumor cells.

By a “photosensitizing agent” or “photosensitizer” is meant a chemical compound that associates with one or more types of selected target cells and, when exposed to light of an appropriate waveband, absorbs the light, causing substances to be produced that impair or destroy the target cells. Virtually any chemical compound that is absorbed or bound to a selected target and absorbs light causing the desired therapy to be effected may be used in the lipid particle of the present invention. As will be appreciated by those of ordinary skill in the art, many different photosensitizers are suitable for use in the present invention. A comprehensive listing of photosensitive chemicals may be found in Kreimer-Bimbaum (1989). Photosensitive agents or compounds include, but are not limited to, chlorins, bacteriochlorins, phthalocyanines, porphyrins, purpurins, merocyanines, psoralens, benzoporphyrin derivatives (BPD), and porfimer sodium (Photofrin®) and pro-drugs such as delta-aminolevulinic acid, which can produce photosensitive agents such as protoporphyrin IX. Other suitable photosensitive compounds include ICG, methylene blue, toluidine blue, texaphyrins, and any other agent that absorbs light in a range of about 500 nm to about 1100 nm, about 550 nm to about 1050 nm, about 600 nm to about 1000 nm, about 650 nm to about 950 nm, about 700 nm to about 900 nm, about 750 nm to about 850 nm, and about 800 nm.

7. Vaccine Agents

In one aspect, the particles can be used as vaccines or in association with antigen delivery. In one embodiment, a patient is immunized with particles having a vaccine agent. In one embodiment, the particles include inactivated vaccines, live vaccines, toxoid vaccines, protein subunit vaccines, polysaccharide vaccines, conjugate vaccines, recombinant vaccines, nucleic acid vaccines, and synthetic vaccines. An inactivated vaccine agent can be a previously virulent micro-organism that has been killed by chemical treatment or heat. Suitable inactivated vaccines include without limitation anthrax, Japanese encephalitis, rabies, polio, diphtheria, tetanus, acellular Pertussis vaccine influenza, cholera, bubonic plague, chicken pox, hepatitis A, Haemophilus influenzae type b, and any combinations thereof.

An attenuated vaccine agent can be a live microorganism modified or cultivated under attenuating conditions that rendered them non-virulent. Suitable inactivated vaccines include without limitation vaccines for chicken pox, yellow fever, measles, rubella, mumps, typhoid, and combinations thereof.

A toxoid vaccine agent can be a toxic compound produced by a microorganism that has been rendered non-toxic. Suitable toxoid vaccines include without limitation vaccines for tetanus, diphtheria, and pertussis. The tetanus vaccine is derived from the toxin called tentanospasmin produced by Clostridium tetani.

A subunit vaccine agent can be a purified antigenic determinant separate from a pathogen. For example, the subunit of the protein coat of a virus, such as the hepatitis B virus. Generally, viral subunit vaccines are free of viral nucleic acids.

Vaccines have been derived from purified forms of the bacterial outer polysaccharide coat. In particular, vaccines for meningitis have been developed using this approach. For example, the purified polyribosylribitol phosphate (PRP) polysaccharide from the capsule of Haemophilus influenzae type b (Hib) has been purified and used as a vaccine (PRP vaccine).

A conjugate vaccine agent is typically an antigenic portion and a polysaccharide portion. These conjugate vaccine agents may also be referred to as polysaccharide conjugate vaccine agents. Suitable conjugate vaccines agents include without limitation those vaccines developed to prevent meningitis. For example the PRP polysaccharide of Hib has been used to develop conjugate vaccine agents (Heath, 1998). It has been linked to diphtheria toxoid (PRP-D), a diphtheria-like protein (PRP-HbOC), a tetanus-toxoid (PRP-T), or a meningococcal outer membrane protein (PRP-OMP). Conjugate vaccine agents for pneumococcal meningitis caused by Streptococcus pneumoniae have also been developed, such as PCV7 (Prevnar) which contains seven different polysaccharides from seven strains of the bacteria known to cause the disease. In PCV7, each polysaccharide is coupled to CRM197, a nontoxic diphtheria protein analogue. For meningitis caused by Neisseria meningitidis, a number of conjugate vaccine agents have been developed, including a polysaccharide (A/C/Y/W-135) diphtheria conjugate vaccine (Menactra) and a monovalent serogroup C glycoconjugate vaccine (MenC).

In certain aspects a vaccine includes a nucleic acid vaccine. The nucleic acid vaccine may be a DNA vaccine, which may be single genes or combinations of genes. Naked DNA vaccines are generally known in the art. (Brower, 1998). Methods for the use of genes as DNA vaccines are well known to one of ordinary skill in the art, and include placing a gene or a portion of a gene under the control of a promoter for expression in a patient in need of treatment. Suitable nucleic acid vaccines include without limitation vaccines for malaria, influenza, herpes, and HIV.

Other drugs or therapeutic compounds, molecules and/or agents include compounds or molecules that affect the central nervous system, such as those affecting neurotransmitters or neural ion channels (i.e. antidepressants (bupropion)), selective serotonin 2c receptor agonists, anti-seizure agents (topiramate, zonisamide), some dopamine antagonists, and cannabinoid-1 receptor antagonists (rimonabant)); leptin/insulin/central nervous system pathway agents (i.e. leptin analogues, leptin transport and/or leptin receptor promoters, ciliary neurotrophic factor (Axokine), neuropeptide Y and agouti-related peptide antagonists, proopiomelanocortin, cocaine and amphetamine regulated transcript promoters, alpha-melanocyte-stimulating hormone analogues, melanocortin-4 receptor agonists, protein-tyrosine phosphatase-1B inhibitors, peroxisome proliferator activated receptor-gamma receptor antagonists, short-acting bromocriptine (ergoset), somatostatin agonists (octreotide), and adiponectin); gastrointestinal-neural pathway agents (i.e. agents that increase glucagon-like peptide-1 activity (extendin-4, liraglutide, dipeptidyl peptidase IV inhibitors), protein YY3-36, ghrelin, ghrelin antagonists, amylin analogues (pramlintide)); and compounds or molecules that may increase resting metabolic rate “selective” beta-3 stimulators/agonist, melanin concentrating hormone antagonists, phytostanol analogues, functional oils, P57, amylase inhibitors, growth hormone fragments, synthetic analogues of dehydroepiandrosterone sulfate, antagonists of adipocyte 11 B-hydroxysteroid dehydrogenase type 1 activity, corticotropin-releasing hormone agonists, inhibitors of fatty acid synthesis, carboxypeptidase inhibitors, and gastrointestinal lipase inhibitors (ATL962).

B. Diagnostic agents

In certain aspects, a particle of the invention can include an imaging or diagnostic agent. Thus, a particle can comprise an MRI agent, an optical agent, an ultrasound agent, etc.

The most commonly employed radionuclide imaging agents include radioactive iodine and indium. Imaging by CT scan may employ a heavy metal such as iron chelates. MRI scanning may employ chelates of gadolinium or manganese. In certain aspects a chelator for a radionuclide is useful for radiotherapy or imaging procedures. Radionuclides useful within the present invention include gamma-emitters, positron-emitters, Auger electron-emitters, X-ray emitters and fluorescence-emitters, with beta- or alpha-emitters preferred for therapeutic use. Examples of radionuclides include: ³²P, ³³P, ⁴³K, ⁴⁷Sc, ⁵²Fe, ⁵⁷Co, ⁶⁴Cu, ⁶⁷Ga, Cu, ⁶⁸Ga, ⁷¹Ge, ⁷⁵Br, ⁷⁶Br, ⁷⁷Br, ⁷⁷As, ⁷⁷Br, ⁸¹Rb/⁸¹K, ⁸⁷MSr, ⁹⁰Y, ⁹⁷Ru, ⁹⁹Tc, ¹⁰⁰Pd, ¹⁰¹Rh, ¹⁰³Pb, ¹⁰⁵Rh, ¹⁰⁹Pd, ¹¹¹Ag, ¹¹¹In, ¹¹³In, ¹¹⁹Sb, ¹²¹Sn, ¹²³I, ¹²⁵I, ¹²⁷Cs, ¹²⁸Ba, ¹²⁹Cs, ¹³¹I, ¹³¹Cs, ¹⁴³Pr, ¹⁵³Sm, ¹⁶¹Tb, ¹⁶⁶Ho, ¹⁶⁹Eu, ¹⁷⁷Lu, ¹⁸⁶Re, ¹⁸⁸Re, ¹⁸⁹Re, ¹⁹¹Os, ¹⁹³Pt, ¹⁹⁴Ir, ¹⁹⁷Hg, ₁₉₉Au, ²⁰³Pb, ²¹¹At, ²¹²Pb, ²¹²Bi and ²¹³Bi. Conditions under which a chelator will coordinate a metal are described, for example, in U.S. Pat. Nos. 4,831,175, 4,454,106 and 4,472,509, each of which is incorporated herein by reference in its entirety.

Technetium-99m (99mTc) is a particularly attractive radioisotope for therapeutic and diagnostic applications, as it is readily available to all nuclear medicine departments, is inexpensive, gives minimal patient radiation doses, and has ideal nuclear imaging properties. It has a half-life of six hours which means that rapid targeting of a technetium-labeled antibody is desirable. Accordingly, in certain embodiments, a lipid particle as described herein includes a chelating agent for technium.

In one aspect the imaging agent is a protein, such as a radiolabeled protein. The proteins suitable for imaging may be antibodies, including fragments or portions of antibodies. Such antibodies include without limitation an indium-111 label or a technetium-99m label. In such embodiments, the radiolabeled antibody serves as both a targeting moiety (antibody) as described herein and an imaging agent (radioisotope). In another embodiment, the protein suitable for imaging is a peptide, such as an RGD peptide.

By “medical imaging agent” or “diagnostic agent” or “diagnostic imaging agent” herein is meant an agent that can be introduced into a cell, tissue, organ or patient and provide an image of the cell, tissue, organ or patient. Most methods of imaging make use of a contrast agent of one kind or another. Diagnostic imaging agents include magnetic resonance imaging (MRI) agents, nuclear magnetic resonance (NMR) agents, x-ray imaging agents, optical imaging agents, ultrasound imaging agents and neutron capture therapy agents.

In one embodiment, the nanoparticle includes a detectable label selected from the group consisting of a fluorescent dye, a quantum dot, a quantum barcode, a metallic particle, a radiographic contrast agent, or a magnetic resonance imaging contrast agent, and combinations thereof.

The detectable label facilitates detection of the cancerous tissue and differentiation of cancerous tissue from healthy tissue. The detectable label can be detected using any one of a number of medical imaging technologies including, but not limited to, x-ray, CT scan, x-ray fluoroscopy, fluorescence, or magnetic resonance imaging, and combinations thereof.

In one embodiment, one or more fluorescent dyes are incorporated into the polymeric nanoparticles. Examples of suitable fluorescent dyes include, but are not limited to, fluoroscein, fluoroscein isothiocyanate, rhodamine dyes, coumarin dyes, luciferin, the AlexaFluor™ family of fluorescent dyes produced by Molecular Probes, or the DyLight Fluor™ family of fluorescent dyes is produced by Thermo Fisher Scientific.

Fluorescent dyes can be incorporated by a number of means either during or after formation of nanoparticles. U.S. Pat. Nos. 4,326,008, 4,267,235, 5,073,498, 5,952,131, 4,613,559, 5,395,688, 4,829,101, 4,996,265, 5,723,218, 5,786,219, 5,326,692, 5,573,909, 5,266,497, 4,613,559, 4,487,855, 5,194,300, 4,774,189, 3,790,492, 6,964,747, which are incorporated herein by specific reference in their entirety, discuss various methods for incorporating fluorescent dyes into polymeric particles. Typical methods include but are not limited to copolymerization, partitioning of water-soluble or oil-soluble dyes into particles by cosolubilization of the dye and the monomer materials in various aqueous and non-aqueous solvents, attachment of the dye by functionalization of internal or external particle surfaces, and encapsulation of the dye by swelling the particle after forming and incorporation of the dye into the resulting spaces.

In one embodiment, a quantum dot or a quantum barcode is incorporated into the nanoparticle. Typically the quantum dot or barcode is first provided and subsequently coated with a polymeric material. Methods for manufacturing mondisperse quantum dots and quantum barcodes are known by persons having skill in the art. For example, quantum dots can be synthesized colloidally from precursor compounds dissolved in solutions based on a three component system composed of precursors, organic surfactants, and solvents. Further discussion of colloidal synthesis of quantum dots can be found in Murray (2001), which is incorporated herein by specific reference in its entirety.

A quantum dot typically consists of a semiconductor nanocrystal (e.g., CdSe) surrounded by a passivation shell (e.g., ZnS). Upon absorption of a photon, an electron-hole pair is generated, the recombination of which in about 10-20 ns leads to the emission of a less-energetic photon. This energy, and therefore the wavelength, is dependent on the size of the quantum dot particle (smaller particles emit at a lower wavelength), which can be varied almost at will by controlled-synthesis conditions.

A quantum barcode has properties similar to a quantum dot except that it absorbs a broad spectrum of light and emits a specific pattern of wavelengths that acts as a particular signature or “barcode.” Typically, a quantum barcode is several different types of quantum dots, each having a particular emission spectrum, that are arranged in a multi layered shell or side-by-side fashion. Quantum barcodes are advantageous at least insofar as their emission pattern produces a particular signature that can be easily detected and tracked.

Metallic particles of a number of types can be incorporated into the nanoparticles either by providing metallic nanoparticles particles or preparing them in situ and coating them with one or more of the a polymer materials as discussed above. Suitable examples of metallic particles that are useful as detectable labels include, but are not limited to, ferric iron oxide (Fe₂O₃) and/or other ferric iron compounds, gadolinium metal or gadolinium-containing compounds, barium sulfate (BaSO₄), or nanogold particles, and combinations thereof.

In one embodiment, the nanoparticles include a detectable label that is a radiographic contrast agent. Radiographic contrast agent can, for example, allow for x-ray imaging of soft tissues such as gastric tissues. In the presently disclosed embodiments, the radiographic contrast agent is included to permit medical personnel to distinguish between cancerous and healthy gastric tissue. Suitable examples of radiographic contrast agents that can be incorporated into nanoparticles include, but are not limited to, barium sulfate (BaSO₄) nanoparticles, nanogold particles, iodine-based x-ray contrast agents, and other materials that include heavy nuclei that efficiently absorb x-rays.

In one embodiment, the nanoparticles include a detectable label that is a nuclear magnetic resonance imaging (MRI) contrast agent. While MRI is typically quite useful for imaging soft tissues, the use of contrast agents is common when imaging the GI tract because it can be difficult to distinguish between the GI tract and the other abdominal organs. In the presently disclosed embodiments, MRI contrast agents are included to permit medical personnel to distinguish between cancerous and healthy gastric tissue. Suitable examples of MRI contrast agents include, but are not limited to, ferric iron oxide (Fe₂O₃) and/or other ferric iron compounds, gadolinium metal or gadolinium-containing compounds, materials containing protons in —CH2— groups, and compounds containing MRI active nuclei that are not naturally abundant in the body, such as helium-3, carbon-13, fluorine-19, oxygen-17, sodium-23, phosphorus-31, and xenon-129.

Ferric iron and gadolinium compounds are paramagnetic agents that shorten the proton spin relaxation times in surrounding water molecules. Materials containing protons in —CH2— groups relax at a faster rate than in water resulting in detectable change in the MRI signal. In another embodiment, the medical imaging agent is an ultrasound agent. By “ultrasound agent” herein is meant an agent that can be used to generate an ultrasound image. Generally, for ultrasound, air in small bubble-like cells, i.e. particles are used as a contrast agent. See U.S. Pat. Nos. 6,219,572, 6,193,951, 6,165,442, 6,046,777, 6,177,062, all of which are hereby expressly incorporated by reference.

C. Targeting moiety

In one embodiment, a targeting moiety is added to the composition. By “targeting moiety” herein is meant a functional group which serves to target or direct the complex to a particular location, cell type, diseased tissue, or association. In general, the targeting moiety is directed against a target molecule and allows concentration of the compositions in a particular localization within a patient. In some embodiments, the agent is partitioned to the location in a non-1:1 ratio. Thus, for example, antibodies, cell surface receptor ligands and hormones, lipids, sugars and dextrans, alcohols, bile acids, fatty acids, amino acids, peptides and nucleic acids may all be attached to localize or target the nanoparticle compositions to a particular site.

In other embodiments, the targeting moiety is a peptide. For example, chemotactic peptides have been used to image tissue injury and inflammation, particularly by bacterial infection; see WO 97/14443, hereby expressly incorporated by reference in its entirety. Peptides may be attached via the chemical linkages to reactive groups on the exterior surface of the lipid particle (Flenniken et al. 2005; Flenniken et al. 2003; Gillitzer et al., 2002; Hermanson, 1996; Wang et al., 2002a; Wang et al., 2002b; Wang et al., 2002c). In some embodiments, peptides are attached to endogenous or engineered reactive functional groups on the exterior surface of each of the lipid particles.

In another embodiment, the peptide is attached to a lipid particle by the mechanism known as “click chemistry” (see Hartmuth et al., 2001). Click chemistry is a modular protocol for organic synthesis that utilizes powerful, highly reliable and selective reactions for the rapid synthesis of compounds. In one application involves the use of azides or alkynes as building blocks due to their ability to react with each other in a highly efficient and irreversible spring-loaded reaction. In one embodiment, the attachment to a lipid particle of (i) proteins as targeting moieties and/or therapeutic agents and/or (ii) drugs as therapeutic agents, is achieved through the use of an azide linkage. In one other embodiment, the attachment of proteins is achieved by a form of peptide ligation utilizing an alkyne-azide cycloaddition reaction (Aucagne et al., 2006).

### II. AMINO ACID-LIPID ANALOGS

Various embodiments of the invention provide methods and compositions related to the synthesis and use of fatty asparagine, serine and cysteine derivatives or a carboxylate salt thereof (also referred to as asparagine, serine, or cysteine derived lipopeptide (ALA, SLA or CLA, respectively) or lipoasparagine, liposerine or lipocysteine) see U.S. Pat. No. 7,439,250, which is incorporated in its entirety herein by reference. It is known that the acid salt of asparagine may be condensed with acetone to form a corresponding pyrimidone (Hardy and Samworth, 1977). In certain embodiments of the invention, a similar reaction is used with the exception that an aldehyde is used in place of acetone (Chu et al., 1992) followed by a subsequent reaction with a chloroformate. In certain embodiments of the invention, the aldehyde and chloroformate will typically contain, independent of each other, an R1 or R2 group, respectively. The composition of the aldehyde and chloroformate dictate the R1 and R2 groups incorporated into the exemplary fatty asparagine derivative (General Formula 1 or a carboxylic salt thereof). In certain embodiments serine and cysteine may be substituted for asparagine without alteration of the chemistry described herein.

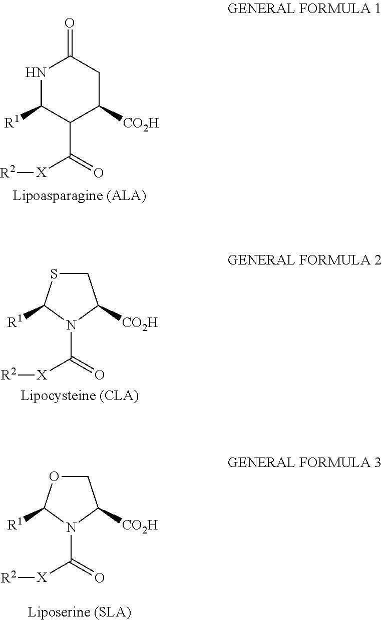

The R1 and R2 may be the same or different moieties. R1 may be a linear, branched, saturated and/or unsaturated hydrocarbon of 5 or more carbon atoms. The hydrocarbon may be 5, 6, 7, 8, 9, 10, 11, 12, 13, 14,15, 16, 17, 18, 19, 20, 21, 22, 23, 24, 25, 30, 35, or more carbon units in length. R1 and/or R2 may also include other derivatives such as cholesterol, steroids, aromatic groups and other hydrophobic molecules or molecules containing hydrophobic groups or derivatives thereof. R2 may be a linear, branched, saturated and/or unsaturated hydrocarbon of 5 or more carbon atoms. The hydrocarbon may be 5, 6, 7, 8, 9, 10, 11, 12, 13, 14,15, 16, 17, 18, 19, 20, 21, 22, 23, 24, 25, 30, 35, or more carbon units in length. R1 and/or R2 may also include other derivatives such as cholesterol, steroids, phenyl, and other hydrophobic molecules or molecules containing hydrophobic groups.

1. Synthesis of Fatty Asparagine Derivatives.

In various embodiments of the invention, an asparagine is typically cyclized with a fatty aldehyde R1CHO, where R1 as described herein. The cyclized amino acid is reacted with a fatty acid chloride or fatty chloroformate, R2XCOCl, where R2 may be any of the groups described above and X is typically an oxygen or a CH₂.

For example, perhydropyrimidinones are synthesized according to the following exemplary scheme (Scheme 1).

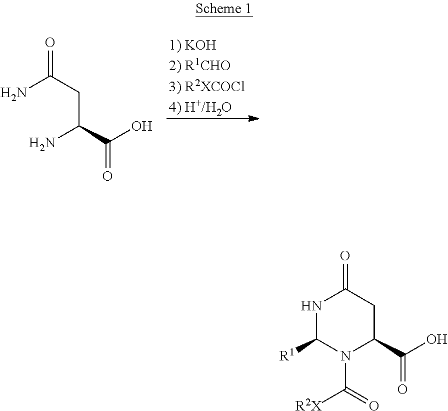

Briefly, D- or L-Asparagine monohydrate (1.0 g, 6.7 mmol) is dissolved in KOH/MeOH solution (10 mL, 0.67 N) and treated with fatty aldehyde (1.0 equiv) for 24 hours at room temperature. Methanol is then removed by using a rotovap and high vacuum. The residue is suspended in 10 mL dioxane/20 mL 10% aqueous Na₂CO₃, stirred well, and chilled in an ice bath. With vigorous stirring, fatty acid chloride or chloroformate (1.0 equiv) dissolved in 10 mL dioxane is added by syringe dropwise. The reaction mixture is allowed to warm slowly to room temperature. After 16 hours, the solution is cooled in an ice bath and 3 mL 10% HCl is added slowly.

At this point, the product may precipitate. In some embodiments, it may be vacuum filtered and rinsed on a Buchner funnel with ice cold water and air dried.

If precipitation does not occur, the acidic solution may be extracted 3×20 mL with Et₂O. Combined organic layers are washed with brine, dried (Na₂SO₄), filtered and the solvents are evaporated. The residue is dried in a round bottom flask on high vacuum.

Flash chromatography may also be performed using 40 g silica gel and eluting with chloroform.

In various embodiments, an co-amino acid-tethered ALAs may be used to (1) form liposomes, (2) modulate liposome properties based on ALA structure, and (3) may be appended after liposomal formation with peptides ligands at the acid group. For example a human growth hormone (hGH) sequence may be appended and used as a peptide ligand. Additional examples of targeting ligands includes, but is not limited to hormones, antibodies, cell-adhesion molecules, saccharides, drugs, and neurotransmitters. Exemplary targeting ligands are described in U.S. Pat. No. 6,287,857 (incorporated herein by reference). In addition, liposomes may bind to receptors and tissues using hGH surface-modified liposomes and RGD-modified liposomes, respectively, or other ligands. An ω-amino acid-tethered ALAs is an asparagine derivative that has been modified with a linker moiety to which other substituents may be coupled.

In certain embodiments, tethering molecules may be incorporated into the asparagine derivatives (compounds such as NH(CH₂)nCO₂H and NH(CH₂CONH)mCH₂CO₂H, where n and m are 1, 2, 3, 4, 5, 6, or more). GABA (4-aminobutanoic acid) may be attached under aqueous conditions using known peptide or protein coupling reagents, such as various bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis(p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene) (for example methods see Corbett and Gleason, 2002). This conversion generates an ALA that is appended with a tether for various modifications such as surface modification for providing enhanced liposomal recognition, which may increase targeting efficiency of liposomes. Various compounds may be used as tethering molecules including, but not limited to amino acids, polypeptides, diamines, polyamines, amino alcohols, amino thiols, diols (e.g., oxiranes for linkage to the heterocycle carboxylic acid to provide an ester and ether linkage from a lipopeptide to a targeting group or other compound), thiol alcohol (e.g., using episulfide for linkage to the heterocycle carboxylic acid to provide an ester linkages at a lipopeptide and a sulfur atom coupling to a targeting group or other compound), dithiols, or a combination thereof (for exemplary methods see U.S. Pat. No. 6,309,842 and Gianolio and McLaughlin, 2001).

### III. ADMINISTRATION

In the methods of the present invention, the lipid compositions can be delivered or administered to a mammal, e.g., a human patient or subject or in the form of a pharmaceutical composition where the lipid compositions are mixed with suitable carriers or excipient(s) in a therapeutically effective amount. By a “therapeutically effective dose”, “therapeutically effective amount”, or, interchangeably, “pharmacologically acceptable dose” or “pharmacologically acceptable amount”, it is meant that a sufficient amount of the composition of the present invention will be present or administered in order to achieve a desired result.

The lipid compositions that are used in the methods of the present invention can be incorporated into a variety of formulations for therapeutic administration. More particularly, the lipid compositions can be formulated into pharmaceutical compositions by combination with appropriate, pharmaceutically acceptable carriers or diluents, and can be formulated into preparations in semi-solid, liquid or gaseous forms; such as capsules, powders, granules, gels, slurries, ointments, solutions, suppositories, injections, inhalants and aerosols. As such, administration of the lipid compositions can be achieved in various ways, including oral, buccal, rectal, parenteral, intraperitoneal, intradermal, transdermal, intratracheal administration. Moreover, the lipid compositions can be administered in a local rather than systemic manner, in a depot or sustained release formulation.

In addition, the lipid compositions can be formulated with common excipients, diluents or carriers, and compressed into tablets, or formulated as elixirs or solutions for convenient oral administration, or administered by the intramuscular or intravenous routes. The lipid compositions can be administered transdermally, and can be formulated as sustained release dosage forms and the like. Lipid compositions can be administered alone, in combination with each other, or they can be used in combination with other known compounds (discussed supra).

Suitable formulations for use in the present invention are found in Remington's Pharmaceutical Sciences (1985), which is incorporated herein by reference. Moreover, for a brief review of methods for drug delivery, see, Langer (1990), which is incorporated herein by reference. The pharmaceutical compositions described herein can be manufactured in a manner that is known to those of skill in the art, i.e., by means of conventional mixing, dissolving, granulating, dragee-making, levigating, emulsifying, encapsulating, entrapping or lyophilizing processes. The following methods and excipients are merely exemplary and are in no way limiting.

For oral administration, the lipid compositions can be formulated readily by combining with pharmaceutically acceptable carriers that are well known in the art. Such carriers enable the compounds to be formulated as pills, capsules, emulsions, lipophilic and hydrophilic suspensions, liquids, gels, syrups, slurries, suspensions and the like, for oral ingestion by a patient to be treated. Pharmaceutical preparations for oral use can be obtained by mixing the lipid compositions with an excipient and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP).

For administration by inhalation, the lipid compositions for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas, or from propellant-free, dry-powder inhalers. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The lipid compositions can be formulated for parenteral administration by injection, e.g., by bolus injection or continuous infusion. Formulations for injection can be presented in unit dosage form, e.g., in ampules or in multidose containers, with an added preservative. The compositions can take such forms as suspensions, solutions or emulsions in oily or aqueous vehicles, and can contain formulator agents such as suspending, stabilizing and/or dispersing agents.

The lipid compositions can also be formulated in rectal compositions such as suppositories or retention enemas, e.g., containing conventional suppository bases such as cocoa butter, carbowaxes, polyethylene glycols or other glycerides, all of which melt at body temperature, yet are solidified at room temperature.

In addition to the formulations described previously, the compounds can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

Liposomes and emulsions are well known examples of delivery vehicles or carriers for hydrophobic drugs. In a presently preferred embodiment, long-circulating, i.e., stealth, liposomes can be employed. Such liposomes are generally described in U.S. Pat. No. 5,013,556, the teaching of which is hereby incorporated by reference. The compounds of the present invention can also be administered by controlled release means and/or delivery devices such as those described in U.S. Pat. Nos. 3,845,770; 3,916,899; 3,536,809; 3,598,123; and 4,008,719; the disclosures of which are hereby incorporated by reference.

Pharmaceutical compositions suitable for use in the present invention include lipid compositions wherein the active ingredients are contained in a therapeutically effective amount. The amount of composition administered will, of course, be dependent on the subject being treated, on the subject's weight, the severity of the affliction, the manner of administration and the judgment of the prescribing physician. Determination of an effective amount is well within the capability of those skilled in the art, especially in light of the detailed disclosure provided herein.

For any compound used in the method of the present invention, a therapeutically effective dose can be estimated initially from cell culture assays or animal models.

Moreover, toxicity and therapeutic efficacy of the compounds described herein can be determined by standard pharmaceutical procedures in cell cultures or experimental animals, e.g., by determining the LD50, (the dose lethal to 50% of the population) and the ED50 (the dose therapeutically effective in 50% of the population). The dose ratio between toxic and therapeutic effect is the therapeutic index and can be expressed as the ratio between LD50 and ED50. Compounds which exhibit high therapeutic indices are preferred. The data obtained from these cell culture assays and animal studies can be used in formulating a dosage range that is not toxic for use in human. The dosage of such compounds lies preferably within a range of circulating concentrations that include the ED50 with little or no toxicity. The dosage can vary within this range depending upon the dosage form employed and the route of administration utilized. The exact formulation, route of administration and dosage can be chosen by the individual physician in view of the patient's condition. (See, e.g., Fingl et al., 1975).

The amount of active compound that can be combined with a carrier material to produce a single dosage form will vary depending upon the disease treated, the mammalian species, and the particular mode of administration. However, as a general guide, suitable unit doses for the compounds of the present invention can, for example, preferably contain between 100 mg to about 3000 mg of the active compound. A preferred unit dose is between 500 mg to about 1500 mg. A more preferred unit dose is between 500 to about 1000 mg. Such unit doses can be administered more than once a day, for example 2, 3, 4, 5 or 6 times a day, but preferably 1 or 2 times per day, so that the total daily dosage for a 70 kg adult is in the range of 0.1 to about 250 mg per kg weight of subject per administration. A preferred dosage is 5 to about 250 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs which have previously been administered; and the severity of the particular disease undergoing therapy, as is well understood by those of skill in the area.

It can be necessary to use dosages outside these ranges in some cases as will be apparent to those skilled in the art. Further, it is noted that the clinician or treating physician will know how and when to interrupt, adjust, or terminate therapy in conjunction with individual patient response.

### IV. EXAMPLES

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

General. Melting points were measured using a Mel-Temp® melting point apparatus and not corrected. The ¹H and ¹³C NMR spectra were obtained using a Varian Inova™ 500 NMR spectrometer operating at 500 and 125 MHz, respectively. All NMR spectra were recorded in chloroform-d or DMSO-d6 as indicated, using tetramethylsilane (TMS) at δ 0.00 for ¹H and residual CDCl₃ at δ 77.00 for ¹³C internal standards. Specific rotations ([α]589) of all novel lipids (1a-g; 9.0 mM solutions in methanol) were determined at room temperature (22° C.) using an AUTOPOL® IV (Rudolph Research) automatic polarimeter at 589 nm. ATR-FTIR spectra were recorded on a Bruker Equinox 55 infrared spectrometer adapted to a Specac Heated Golden Gate temperature controller. All liposome formulations were prepared using a Lipex™ stainless steel extruder (Northern Lipids Inc., Burnaby, Canada). All optical density (OD) measurements were carried out using a duel diode array CARY 5000 Varian UV-visible spectrophotometer. Scanning electron microscopy (SEM) studies were performed on Hitachi S5500 cold field emission scanning electron microscope operating at 1-30 kV with 1.6-0.4 nm resolution. High resolution optical microscopy was performed using Axio Scope 40 POL polarizing microscope (Carl Zeiss Microimaging, Inc., Thornwood, N.Y.).

Material. Disteroylphosphatidylcholine (DSPC) was purchased from Avanti Polar Lipids, Alabaster, Ala. All other chemicals (dodecanal, decanoyl chloride, L-asparagine, etc.) were obtained from either Sigma-Aldrich or Acros Organics. Dubecos phosphate buffer saline (PBS; pH 7.4), polycarbonate filters for extrusion, and Electron Microscopy Diatome copper grids with formvar/carbon Film (400 mesh) for electron microscopy were purchased from Fischer Scientific. All chemicals were reagent grade and were used as received.

General procedure for the synthesis of lipids ALA_{n,m} (1a-g). L-Asparagine (1.0 mmol) and sodium hydroxide (1.0 mmol) were added to methanol (10 mL) and the mixture was stirred for 15 min. To this clear solution, fatty aldehyde (1.2 mmol in 10 mL of methanol) was added and the mixture stirred overnight at room temperature. The methanol was evaporated and the residue was washed with hexane (3×, 25 mL). The resulting white powder and 2,6-lutidine (1.1 mmol) were taken up in THF (20 mL), cooled to 0° C., and the fatty acyl chloride (1.1 mmol in 10 mL of THF) was added slowly (in 15 min). These processes were performed in a capped vessel with a small vent to reduce atmospheric exposure. After stirring overnight, the reaction mixture was poured into 10% HCl (100 mL) and the precipitated crude product was either separated by filtration or extracted with dichloromethane to yield an off-white solid. These solids were triturated (ethyl acetate/hexanes) to yield products as white amorphous powders.

3-Hexanoyl-6-oxo-2-pentylhexahydropyrimidine-4-carboxylic acid (ALA_{5,5}; 1a). The title compound was prepared from asparagine, hexanal, and hexanoyl chloride, and extracted with dichloromethane to obtain an off-white solid after trituration in 91% yield; mp: 55-56° C.; [α]⁵⁸⁹: −72.7 (2.81 g/L, MeOH); ¹H NMR (500 MHz, CDCl₃): δ 0.83-0.93 (m, 6H, 2CH₃), 1.24-1.41 (m, 10H, 5CH₂), 1.56-1.71 (m, 2.40H, anti-H-1″ & 2H-3′), 1.77-1.89 (m, 1.60H, anti-H-1″ & 2syn-H-1″), 2.35 (dt, J=7.8, 16.1 Hz, 1.40H, syn-H-2′ & 2anti-H-2′), 2.45 (dt, J=7.8, 15.6 Hz, 0.60H, syn-H-2′), 2.78 (dd, J=7.3, 17.6 Hz, 0.40H, anti-H-5), 2.82 (dd, J=8.3, 17.6 Hz, 0.60H, syn-H-5), 2.98 (dd, J=6.4, 17.1 Hz, 0.40H, anti-H-5), 2.99 (dd, J=9.8, 17.1 Hz, 0.60H, syn-H-5), 4.73 (t, J=6.8 Hz, 0.40H, anti-H-4), 4.99 (t, J=8.8 Hz, 0.60H, syn-H-4), 5.11 (dt, J=4.9, 9.8 Hz, 0.60H, syn-H-2), 5.76-5.82 (m, 0.40H, anti-H-2), 7.80 (br d, 0.40H, anti-H-1), 8.27 (br d, J=4.4 Hz, 0.60H, syn-H-1); ¹³C NMR (125 MHz, CDCl₃): δ 13.8 (q), 22.4 (t), 24.6 (t), 24.8 (t, anti), 25.2 (t, syn), 31.01 (t), 31.04 (t), 31.09 (t), 31.3 (t), 31.4 (t, anti), 33.0 (t, syn), 33.3 (t, anti), 35.4 (t, anti), 37.1 (t, syn), 51.1 (d, syn), 52.1 (d, anti), 62.8 (d, anti), 65.6 (d, syn), 169.9 (s, anti), 170.6 (s, syn), 172.3 (s, syn), 172.5 (s, anti), 173.7 (s, syn), 174.1 (s, anti); ATR-FTIR (νₘₐₓ, cm⁻¹): 3240, 2924, 2857, 1719, 1632, 1399, 1189; MS (CID, m/z): 312.9 (100, MH⁺), 213.9 (27, MH⁺—C₅H₁₀CHO), 100.0 (50, C₅H₁₀CHO⁺); Exact mass analysis: calcd for C₁₆H₂₈N₂O₄ (MH⁺) 313.2127, found 313.2105.

3-Dodecanoyl-6-oxo-2-pentylhexahydropyrimidine-4-carboxylic acid (ALA_{5,11}; 1b). The title compound was prepared from asparagine, hexanal, and dodecanoyl chloride, and extracted with dichloromethane to obtain an off-white solid after trituration in 78% yield; mp: 118-120° C.; [α]⁵⁸⁹: −62.5 (3.57 g/L, MeOH); ¹H NMR (500 MHz, CDCl₃): δ 0.88 (t, J=7.3 Hz, 3H, CH₃), 0.90 (t, J=6.8 Hz, 3H, CH₃), 1.21-1.41 (m, 22H, 11CH₂), 1.56-1.71 (m, 2.36H, anti-H-1″ & 2H-3′), 1.77-1.89 (m, 1.64H, anti-H-1″ & 2syn-H-1″), 2.31-2.37 (m, 0.72H, 2anti-H-2′), 2.36 (dt, J=7.8, 15.6 Hz, 0.64H, syn-H-2′), 2.45 (dt, J=7.8, 15.6 Hz, 0.64H, syn-H-2″), 2.78 (dd, J=7.8, 17.1 Hz, 0.36H, anti-H-5), 2.81 (dd, J=8.3, 17.1 Hz, 0.64H, syn-H-5), 2.98 (dd, J=5.9, 17.1 Hz, 0.36H, anti-H-5), 3.04 (dd, J=9.8, 17.1 Hz, 0.64H, syn-H-5), 4.73 (t, J=6.8 Hz, 0.36H, anti-H-4), 5.00 (t, J=9.3 Hz, 0.64H, syn-H-4), 5.10 (dt, J=5.4, 9.3 Hz, 0.64H, syn-H-2), 5.75-5.81 (m, 0.36H, anti-H-2), 7.66 (br d, 0.36H, anti-H-1), 8.10 (br d, J=4.4 Hz, 0.64H, syn-H-1); ¹³C NMR (125 MHz, CDCl₃): δ 13.9 (q), 14.1 (q), 22.4 (t, anti), 22.6 (t, syn), 24.8 (t, syn), 25.3 (t, anti), 29.27 (t), 29.29 (t), 29.38 (t), 29.46 (t), 29.57 (t), 29.59 (t), 30.96 (t), 31.07 (t), 31.12 (t), 31.5 (t), 31.9 (t), 33.1 (t, syn), 33.5 (t, anti), 35.5 (t, anti), 37.1 (t, syn), 51.1 (d, syn), 52.1 (d, anti), 62.9 (d, anti), 65.7 (d, syn), 169.9 (s, anti), 170.6 (s, syn), 172.3 (s, anti), 172.4 (s, syn), 174.0 (s, syn), 174.4 (s, anti); ATR-FTIR (νₘₐₓ, cm⁻¹): 3224, 2923, 2855, 1734, 1633, 1392, 1215; MS (CID, m/z): 396.9 (100, MH⁺), 297.8 (20, MH⁺—C₅H₁₀CHO), 102.2 (40, C₆H₁₃OH⁺); Exact mass analysis: calcd for C₂₂H₄₀N₂O₄ (MH⁺) 397.3066, found 397.3042.

3-Hexanoyl-6-oxo-2-undecylhexahydropyrimidine-4-carboxylic acid (ALA_{11,5}; 1c). The title compound was prepared from asparagine, dodecanal, and hexanoyl chloride, and extracted with dichloromethane to obtain an off-white solid after trituration in 82% yield; mp: 106-107° C.; [α]⁵⁸⁹: −62.3 (3.57 g/L, MeOH); ¹H NMR (500 MHz, CDCl₃): δ 0.88 (t, J=6.9 Hz, 3H, CH₃), 0.91 (t, J=6.4 Hz, 3H, CH₃), 1.22-1.39 (m, 22H, 11CH₂), 1.56-1.73 (m, 2.33H, anti-H-1″ & 2H-3′), 1.78-1.89 (m, 1.67H, anti-H-1″ & 2syn-H-1″), 2.34 (t, J=7.3 Hz, 0.67H, 2anti-H-2′), 2.36 (dt, J=7.8, 15.6 Hz, 0.67H, syn-H-2′), 2.45 (dt, J=7.8, 15.6 Hz, 0.67H, syn-H-2′), 2.79 (dd, 0.33H, anti-H-5), 2.81 (dd, J=8.3, 17.1 Hz, 0.67H, syn-H-5), 2.98 (dd, J=5.9, 17.1 Hz, 0.33H, anti-H-5), 3.05 (dd, J=9.8, 17.1 Hz, 0.67H, syn-H-5), 4.72 (t, J=6.8 Hz, 0.33H, anti-H-4), 4.99 (t, J=9.0 Hz, 0.67H, syn-H-4), 5.09 (dt, J=4.9, 9.8 Hz, 0.67H, syn-H-2), 5.75-5.81 (m, 0.33H, anti-H-2), 7.56 (br d, 0.33H, anti-H-1), 8.06 (br d, J=4.9 Hz, 0.67H, syn-H-1); ¹³C NMR (125 MHz, CDCl₃): δ 13.9 (q), 14.1 (q), 22.4 (t), 22.6 (t), 24.5 (t), 25.3 (t, anti), 25.7 (t, syn), 29.0 (t, syn), 29.1 (t, anti), 29.3 (t), 29.4 (t), 29.50 (t), 29.54 (t), 29.56 (t), 29.59 (t), 30.9 (t, syn), 31.4 (t), 31.5 (t, anti), 31.9 (t), 33.1 (t, syn), 33.4 (t, anti), 35.6 (t, anti), 37.2 (t, syn), 51.1 (d, syn), 52.1 (d, anti), 62.9 (d, anti), 65.7 (d, syn), 169.9 (s, anti), 170.7 (s, syn), 172.3 (s, syn), 172.4 (s, anti), 174.0 (s, syn), 174.5 (s, anti); ATR-FTIR (νₘₐₓ, cm⁻¹): 3218, 2922, 2849, 1731, 1633, 1391, 1214; MS (CID, m/z): 397.3 (100, MH⁺), 214.0 (6, MH⁺—C₁₁H₂₂CHO), 184.2 (80, C₁₁H₂₂CHO); Exact mass analysis: calcd for C₂₂H₄₀N₂O₄ (MH⁺) 397.3066, found 397.3041.

3-Dodecanoyl-6-oxo-2-undecylhexahydropyrimidine-4-carboxylic acid (ALA_{11,11}; 1d). The title compound was prepared from asparagine, dodecanal, and dodecanoyl chloride as an off-white solid, filtered, and washed in 74% yield; mp: 110-112° C.; [α]⁵⁸⁹: −44.6 (4.33 g/L, MeOH); ¹H NMR (500 MHz, CDCl₃): δ 0.89 (t, J=6.9 Hz, 6H, 2CH₃), 1.22-1.40 (m, 36H, 18CH₂), 1.56-1.76 (m, 2.32H, anti-H-1″ & 2H-3′), 1.77-1.89 (m, 1.68H, anti-H-1″ & 2syn-H-1″), 2.30-2.36 (m, 0.64H, 2anti-H-2′), 2.36 (ddd, J=7.3, 7.8, 15.6 Hz, 0.68H, syn-H-2′), 2.45 (ddd, J=7.3, 7.8, 15.6 Hz, 0.68H, syn-H-21), 2.79 (dd, 0.32H, anti-H-5), 2.81 (dd, J=8.8, 17.6 Hz, 0.68H, syn-H-5), 2.98 (dd, J=6.4, 17.1 Hz, 0.32H, anti-H-5), 3.08 (dd, J=9.3, 17.1 Hz, 0.68H, syn-H-5), 4.72 (t, J=6.8 Hz, 0.32H, anti-H-4), 4.99 (t, J=9.3 Hz, 0.68H, syn-H-4), 5.09 (dt, J=5.4, 9.3 Hz, 0.68H, syn-H-2), 5.75-5.81 (m, 0.32H, anti-H-2), 7.39 (br d, 0.32H, anti-H-1), 7.88 (br d, J=4.9 Hz, 0.68H, syn-H-1); ¹³C NMR (125 MHz, CDCl₃): δ 14.0 (q), 22.6 (t), 24.8 (t), 25.3 (t, anti), 25.6 (t, syn), 29.0 (t, syn), 29.1 (t, anti), 29.27 (t), 29.29 (t), 29.31 (t), 29.39 (t), 29.45 (t), 29.47 (t), 29.50 (t), 29.55 (t), 29.57 (t), 29.59 (t), 29.61 (t), 31.0 (t, syn), 31.5 (t, anti), 31.9 (t), 33.1 (t, syn), 33.5 (t, anti), 35.6 (t, anti), 37.2 (t, syn), 51.1 (d, syn), 52.1 (d, anti), 62.9 (d, anti), 65.6 (d, syn), 169.9 (s, anti), 170.7 (s, syn), 172.2 (s, syn), 172.3 (s, anti), 174.0 (s, syn), 174.5 (s, anti); ATR-FTIR (νₘₐₓ, cm⁻¹): 3202, 2920, 2849, 1727, 1627, 1468, 1399, 1212; MS (ES, m/z): 504.0 (100, MH+Na⁺), 503.5 (85, M+Na⁺), 184.2 (4, C₁₁H₂₂CHO⁺); Exact mass analysis: calcd for C₂₈H₅₂N₂O₄ (MH⁺) 481.4005, found 481.4013.

6-Oxo-3-stearoyl-2-undecylhexahydropyrimidine-4-carboxylic acid (ALA_{11,17}; 1e). The title compound was prepared from asparagine, dodecanal, and octadecanoyl chloride as an off-white solid, filtered, and washed in 81% yield; mp: 105-109° C.; [α]⁵⁸⁹: −37.8 (5.08 g/L, MeOH); ¹H NMR (500 MHz, CDCl₃): δ 0.88 (t, J=6.8 Hz, 6H, 2CH₃), 1.22-1.39 (m, 46H, 23CH₂), 1.63 (tt, J=6.8, 7.8 Hz, 2H, 2H-3D, 1.62-1.71 (m, 0.67H, 2anti-H-1″), 1.78-1.89 (m, 1.33H, 2syn-H-1″), 2.34 (dd, J=6.3, 7.8 Hz, 0.67H, 2anti-H-2′), 2.35 (dt, J=7.8, 15.6 Hz, 0.67H, syn-H-2′), 2.44 (ddd, J=6.4, 7.3, 14.6 Hz, 0.67H, syn-H-2′), 2.77 (dd, J=7.3, 16.6 Hz, 0.33H, anti-H-5), 2.81 (dd, J=8.3, 17.1 Hz, 0.67H, syn-H-5), 2.98 (dd, J=5.9, 16.6 Hz, 0.33H, anti-H-5), 3.07 (dd, J=9.8, 17.1 Hz, 0.67H, syn-H-5), 4.73 (t, J=6.4 Hz, 0.33H, anti-H-4), 4.97 (t, J=8.8 Hz, 0.67H, syn-H-4), 5.09 (dt, J=5.9, 7.8 Hz, 0.67H, syn-H-2), 5.74-5.80 (m, 0.33H, anti-H-2), 7.37 (br d, 0.33H, anti-H-1), 7.90 (br d, J=4.4 Hz, 0.67H, syn-H-1); ¹³C NMR (125 MHz, CDCl₃): δ 14.3 (q), 22.9 (t), 25.1 (t, syn), 26.0 (t, anti), 29.27 (t), 29.34 (t), 29.37 (t), 29.54 (t), 29.57 (t), 29.60 (t), 29.69 (t), 29.73 (t), 29.79 (t), 29.83 (t), 29.90 (t), 29.92 (t), 29.93 (t), 29.96 (t), 31.2 (t), 31.9 (t), 32.2 (t), 33.4 (t, syn), 33.8 (t, anti), 35.9 (t, anti), 37.4 (t, syn), 51.4 (d, syn), 52.4 (d, anti), 63.2 (d, anti), 66.0 (d, syn), 170.0 (s, anti), 170.8 (s, syn), 172.6 (s, anti), 172.7 (s, syn), 174.2 (s, syn), 174.9 (s, anti); ATR-FTIR (νₘₐₓ, cm⁻¹): 3218, 2918, 2849, 1733, 1631, 1469, 1399, 1211; MS (ES, m/z): 588.6 (100, MH+Na⁺), 587.7 (85, M+Na⁺), 382.2 (20, MH⁺—C₁₁H₂₂CHO); Exact mass analysis: calcd for C₃₄H₆₄N₂O₄ (MH⁺) 565.4944, found 565.4927.

3-Dodecanoyl-2-heptadecyl-6-oxohexahydropyrimidine-4-carboxylic acid (ALA_{17,11}; 1f). The title compound was prepared from asparagine, octadecanal, and dodecanoyl chloride as an off-white solid, filtered, and washed in 67% yield; mp: 104-107° C.; [α]⁵⁸⁹: −42.0 (5.08 g/L, MeOH); ¹H NMR (500 MHz, CDCl₃): δ 0.88 (t, J=6.8 Hz, 6H, 2CH₃), 1.22-1.39 (m, 46H, 23CH₂), 1.63 (quintet, J=7.8 Hz, 2H, 2H-3), 1.63-1.71 (m, 0.67H, 2anti-H-1″), 1.78-1.89 (m, 1.33H, 2syn-H-1″), 2.34 (t, J=7.8 Hz, 0.67H, 2anti-H-2T), 2.36 (dt, J=7.8, 15.6 Hz, 0.67H, syn-H-2′), 2.45 (dt, J=7.8, 15.6 Hz, 0.67H, syn-H-2′), 2.79 (dd, J=8.8, 17.1 Hz, 0.33H, anti-H-5), 2.81 (dd, J=8.3, 17.1 Hz, 0.67H, syn-H-5), 2.98 (dd, J=5.9, 17.1 Hz, 0.33H, anti-H-5), 3.09 (dd, J=9.8, 17.1 Hz, 0.67H, syn-H-5), 4.73 (t, J=6.8 Hz, 0.33H, anti-H-4), 4.99 (t, J=8.9 Hz, 0.67H, syn-H-4), 5.09 (dt, J=5.4, 9.3 Hz, 0.67H, syn-H-2), 5.75-5.81 (m, 0.33H, anti-H-2), 7.36 (br d, 0.33H, anti-H-1), 7.90 (br d, J=4.9 Hz, 0.67H, syn-H-1); ¹³C NMR (125 MHz, CDCl₃): δ 14.1 (q), 22.7 (t), 24.8 (t), 25.4 (t, anti), 25.7 (t, syn), 29.0 (t, syn), 29.1 (t, anti), 29.29 (t), 29.32 (t), 29.34 (t), 29.40 (t), 29.44 (t), 29.48 (t), 29.49 (t), 29.59 (t), 29.60 (t), 29.62 (t), 29.65 (t), 29.70 (t), 29.71 (t), 29.72 (t), 31.0 (t, syn), 31.7 (t, anti), 31.9 (t), 33.2 (t, syn), 33.5 (t, anti), 35.6 (t, anti), 37.2 (t, syn), 51.2 (d, syn), 52.2 (d, anti), 63.0 (d, anti), 65.7 (d, syn), 169.6 (s, anti), 170.4 (s, syn), 172.3 (s, anti), 172.5 (s, syn), 173.6 (s, syn), 174.4 (s, anti); ATR-FTIR (νₘₐₓ, cm⁻¹): 3220, 2918, 2848, 1734, 1664, 1631, 1469, 1400, 1211, 721; MS (ES, m/z): 604.1 (100, M+NaOH⁺), 398.1 (85, M⁺-C₁₂H₂₂); Exact mass analysis: calcd for C₃₄H₆₄N₂O₄ (MH⁺) 565.4944, found 565.4922.

2-Heptadecyl-6-oxo-3-stearoylhexahydropyrimidine-4-carboxylic acid (ALA_{17,17}; 1g). The title compound was prepared from asparagine, octadecanal, and octadecanoyl chloride as an off-white solid, filtered, and washed in 80% yield; mp: 107-111° C.; [α]⁵⁸⁹: N/A (see footnote f in Table 1); ¹H NMR (500 MHz, CDCl₃): δ 0.88 (t, J=6.8 Hz, 6H, 2CH₃), 1.20-1.39 (m, 58H, 29CH₂), 1.57-1.71 (m, 2.32H, anti-H-1″ & 2H-3), 1.78-1.89 (m, 1.68H, anti-H-1″ & 2syn-H-1″), 2.30-2.36 (m, 0.64H, 2anti-H-2t), 2.37 (dt, J=7.3, 15.6 Hz, 0.68H, syn-H-2), 2.45 (dt, J=7.3, 15.6 Hz, 0.68H, syn-H-2′), 2.79 (dd, 0.32H, anti-H-5), 2.80 (dd, J=8.3, 17.1 Hz, 0.68H, syn-H-5), 2.98 (dd, J=6.3, 17.1 Hz, 0.32H, anti-H-5), 3.12 (dd, J=9.8, 17.1 Hz, 0.68H, syn-H-5), 4.73 (t, J=6.8 Hz, 0.32H, anti-H-4), 4.99 (t, J=8.8 Hz, 0.68H, syn-H-4), 5.08 (dt, J=5.9, 8.6 Hz, 0.68H, syn-H-2), 5.74-5.80 (m, 0.32H, anti-H-2), 7.15-7.19 (br s, 0.32H, anti-H-1), 7.62-7.66 (br s, 0.68H, syn-H-1); ¹³C NMR (125 MHz, CDCl₃): δ 14.1 (q), 22.7 (t), 24.9 (t), 25.4 (t, anti), 25.7 (t, syn), 29.02 (t), 29.10 (t), 29.32 (t), 29.35 (t), 29.43 (t), 29.48 (t), 29.53 (t), 29.59 (t), 29.66 (t), 29.72 (t), 31.0 (t, syn), 31.7 (t, anti), 31.9 (t), 33.2 (t, syn), 33.5 (t, anti), 35.6 (t, anti), 37.1 (t, syn), 51.2 (d, syn), 52.2 (d, anti), 63.0 (d, anti), 65.8 (d, syn), 169.6 (s, anti), 170.3 (s, syn), 172.3 (s, anti), 172.6 (s, syn), 173.6 (s, syn), 174.4 (s, anti); ATR-FTIR (νₘₐₓ, cm⁻¹): 3227, 2917, 2849, 1733, 1634, 1470, 1399, 1214; MS (ES, m/z): 688.7 (45, M⁺+NaOH), 686.2 (100, M⁺+NaOH-3H), 398.1 (33, M⁺-C₁₈H₃₃); Exact mass analysis: calcd for C₄₀H₇₆N₂O₄ (MH⁺) 649.5883, found 649.5913.

General procedure for preparation of liposomes. A mixture of DSPC and lipid 1 (5, 10, 25, or 50 mol % 1 and DSPC; total moles lipid: 1.26×10⁻⁴) in a round bottom flask was dissolved in chloroform. The solvent was evaporated under reduced pressure to obtain a thin film and was further stripped of solvent under house vacuum for 45-60 min before storage (4° C.). The thin film was hydrated with 3.5 mL of PBS (10 mM phosphate buffer and 120 mM NaCl, pH 7.4). The resulting emulsion was vortexed and incubated at 55-60° C. alternately until a cloudy suspension of multilamellar vesicles (MLVs) was formed. The MLVs were subjected to sequential extrusion with moderate pressure (200-700 psi) through polycarbonate filters of descending pore sizes (3× through each filter; pore sizes: 2.0, 1.0, 0.40, 0.20, and 0.10 μm) mounted in a stainless steel extruder connected to circulating warm water (60-65° C.). The extrusion procedure produced unilamellar liposomes with a mean diameter of 134 nm after the third extrusion through 0.10 μm filter as determined by DLS analysis. Repeated extrusions (11×) through the 0.10 μm filter produced smaller liposomes (95 nm). For convenience, all liposomes were prepared by the 3× extrusion protocol and used within 24 hours for all experiments unless otherwise noted.

Sample preparation for scanning electron microscopy (SEM) studies. The liposome suspension (50 μL) obtained by the above protocol was diluted with equal volume of 0.1 M non-saline phosphate buffer. The diluted sample was treated with an 11% (w/w; pH 7.2) solution of ammonium molybdate ((NH₄)₆Mo₇O₂₄; 30 μL) and allowed to stand (in the open air) at room temperature for at least 5 h. Samples were prepared by putting drops of this suspension on 300 mesh copper grids coated with either lacey carbon or Formvar and the excess liquid was carefully removed using a pointed filter paper. The copper grids were placed on a filter paper (in a Petri dish) and dried in air for 3 h before SEM analysis. To collect images under acidic conditions, each liposome sample was acidified to the desired pH (3.4 or 1.9), allowed to stand 20-30 min, and stained with (NH₄)₆Mo₇O₂₄. A drop of the solution was added to the grids, the excess liquid was removed by a pointed filter paper, and images were collected immediately.

Results

Lipid synthesis. The linear alkyl groups of ALA_{n,m} (R and R′; Scheme 1) were selected in order to investigate the effect of the fatty chain lengths on liposome formation and properties. The synthesis of prototype ALA_{11,17} was examined in detail due to the commercial availability of corresponding aldehyde and acid chloride components as well as the likelihood that this analogue would be compatible with self-assembled structures. Preliminary synthetic studies were performed by sequentially adding asparagine and dodecanal to basic THF/water mixtures (Lakner and Negrete, 2002) followed by stearoyl chloride addition to obtain ALA_{11,17} in variable yields, ranging from 20-45%. The use of polar organic solvents (DMF, DMSO, THF) with various proportions of triethylamine afforded similar yields. Proton NMR experiments (D₂O or CD₃OH) indicated that the initial cyclocondensation of L-asparagine and the aldehyde (to the tetrahydropyrimidinone intermediate, Scheme 1) occurs in high efficiency suggesting that losses in the amine acylation cause the low yields. The preparation was improved by forming the initial tetrahydropyrimidinone in methanol followed by rigorous solvent removal and acylation in THF/lutidine (Scheme 1) (Erickson et al., 1966; Lynch et al., 1989; Saaidi et al, 2007). This one-pot procedure afforded a waxy solid, which upon trituration (ethyl acetate/hexane) typically gave ALA_{11,17} as a white solid in good to excellent yields (Table 1). Following this protocol, seven novel lipid analogues (1a-g) were synthesized. Among these, only ALA_{17,17} required chromatographic isolation (silica gel, dichloromethane/methanol 9:1). Molecular weights, specific rotations, melting points, and the yields of the products are summarized in Table 1.

All ALAs exhibited ¹H and ¹³C NMR spectroscopic signals consistent with the presence of two product conformers (syn-1 and anti-1; Scheme 2). The Keq values ([syn-1]/[anti-1]; Table 1) generally increased for longer alkyl chains as is expected for increasing van der Waal stabilization of the syn conformer (Mecke et al., 2003; Gruner, 1987; Tanford, 1980). The decreasing specific rotation for lipids with longer chain lengths is correlated with the decreasing molarity of solutions formed from similar weights of analyte.

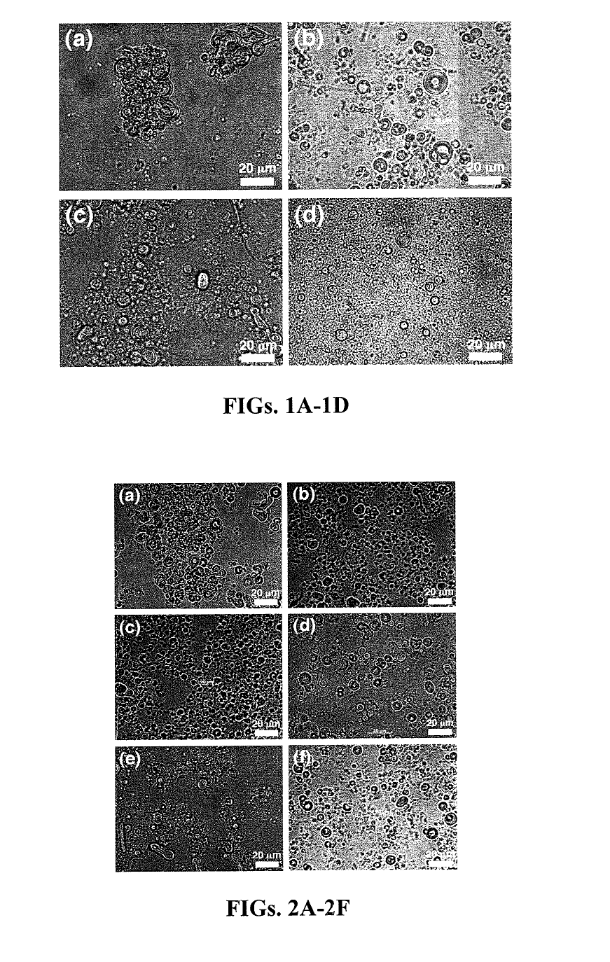

Preparation and characterization of MLVs prepared from DSPC and ALA_{n,m}/DSPC mixtures. Multilamellar vesicle (MLV) suspensions of DSPC, and 5, 10, 15, 25, and 50 mol % ALA_{11,17}/DSPC in phosphate buffer saline (PBS, pH 7.4) were individually prepared by thin film hydration and analyzed by high resolution optical microscopy (HROM; FIG. 1). The HROM image of DSPC MLVs showed the presence of clustered vesicles of varying size (FIG. 1A). In contrast, those prepared using 5, 10, 15, 25, and 50 mol % ALA_{11,17}/DSPC appeared as isolated vesicles (for example, see FIGS. 1B-D).

To examine the effect of chain lengths of novel lipids on the MLV structures and aggregation (Gruner, 1987; Guler et al., 2009; Rosenzaeig et al., 2000; Subczynski et al., 1993), MLV suspensions of 10 mol % ALA_{n,m}/DSPC were prepared and examined by HROM (FIG. 2). The micrograph of 10 mol % ALA_{5,5}/DSPC MLVs (the least lipophilic ALA; FIG. 2A) exhibited smaller and less tightly clustered vesicles compared to the DSPC sample (FIG. 1A). The micrographs of all the other ALA-containing MLVs showed smaller, dispersed MLV structures (FIGS. 2B-F). These data suggest that two short alkyl chains of ALA_{5,5} are not sufficiently long for quantitatively anchoring into the liposome bilayer and thus have a reduced effect on vesicle aggregation. The ALAs bearing at least one undecyl chain (C11), on the other hand, apparently incorporated at greater proportions into the liposome bilayer and significantly influenced vesicle aggregation (FIGS. 2B-F) (Rosenzaeig et al., 2000; Subczynski et al., 1993). At pH 7.4 the net negative surface charge generated by the imbedded ALAs induces repulsions that cause the dispersion of MLVs (MacLachlan, 2006). Thus, 10 mol % or higher composition of ALA_{m,n} with the shorter alkyl chains weakly diminish the propensity of DSPC MLVs to cluster while those with alkyl chains of C11 or greater strongly decrease MLV aggregation.

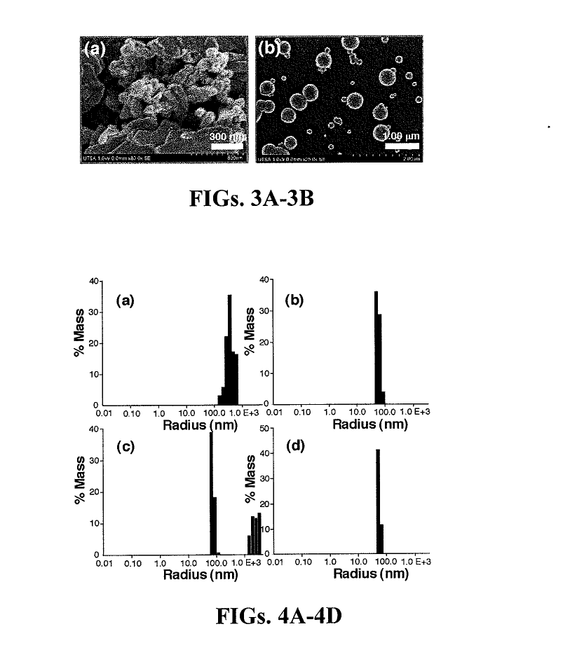

The observation that MLV preparations of ALA_{11,17}/DSPC appeared to form well-defined morphologies as compared to those generated from DSPC alone prompted an investigation of the MLV structures using scanning electron microscopy (SEM). Hence, both DSPC and 10 mol % ALA_{11,17}/DSPC MLVs were freshly prepared, negatively stained ((NH₄)₆Mo₇O₂₄), and visualized by SEM (FIG. 3). The SEM image of the DSPC MLVs exhibited diverse sizes of aggregated structures (FIG. 3A) confirming the finding of HROM studies (FIG. 1A). The SEM image of the sample containing 10 mol % ALA_{11,17}/DSPC (FIG. 3B) exhibited a roughly bimodal size distribution of vesicles with sizes in the ranges of 100-200 and 400-500 nm, respectively. The electron micrograph of unextruded self-assemblies of 100 mol % ALA_{11,17} as control exhibited spherical structures but with less morphological uniformity. The heterocyclic headgroup likely endows ALA_{11,17} with conicity that templates bilayer curvature, which influences the observed size distribution and morphology of the vesicles (Hui, 1993).

Preparation and characterization of DSPC and ALA_{n,m}/DSPC liposomes. The above MLV suspensions (FIGS. 1 and 2) were sequentially extruded using filters of decreasing pore size to generate liposome suspensions for each sample. Dynamic light scattering experiments were conducted to measure the particle size distributions of DSPC and 10 mol % ALA_{11,17}/DSPC liposomes. DSPC liposomes at pH 7.4 displayed a wide size distribution ranging from 250 to 700 nm in diameter (FIG. 4A) consistent with the low colloidal stability of DSPC liposomes. The 10 mol % ALA_{11,17}/DSPC, on the other hand, showed a smaller size and narrower size distribution at the same pH, ranging from 100-250 nm diameter with a calculated mean size of 134 nm (FIG. 4B).

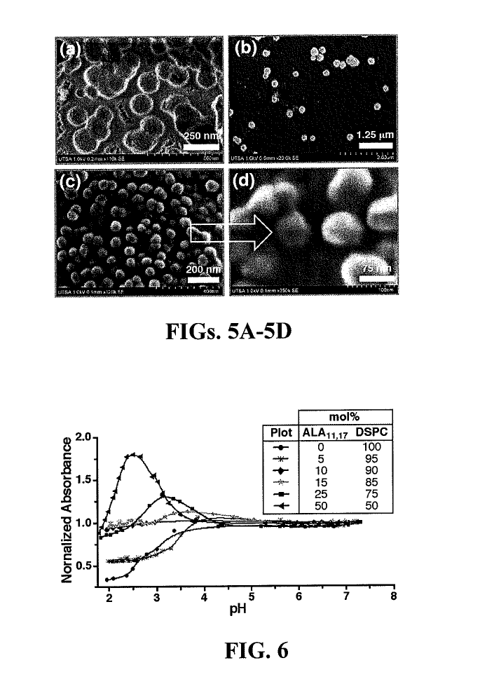

Freshly prepared DSPC and 10 mol % ALA_{11,17}/DSPC liposome samples were negatively stained with (NH₄)₆Mo₇O₂₄ and analyzed by SEM. The electron micrographs showed size distributions similar to those obtained by DLS (FIG. 5). The SEM image of DSPC liposomes at pH 7.4 showed clustered, but unfused nanoparticles (FIG. 5A), though the majority of the observed structures varied widely in morphology and size. This observation together with the clustering of the DSPC microvesicles seen in HROM and SEM images (FIGS. 1A and 3A, respectively) is a consequence of the aggregation of phosphatidylcholine nanostructures bearing neutral surface charge at pH 7.4 (Hui, 1993). In contrast, SEM images of liposomes formulated with 10 mol % ALA_{11,17}/DSPC appeared as isolated spheres at pH 7.4 with size distribution comparable with that found in DLS analysis (FIGS. 5B-D). These samples maintained colloidal homogeneity in ambient conditions for a longer period of time. Self-assembled vesicles composed of ALA_{11,17}/DSPC contain negatively charged surfaces at pH 7.4 (vide supra) and consequently resist aggregation. Thus, ALA_{11,17} contributes to the colloidal stability of ALA_{11,17}/DSPC formulations at pH 7.4.

Acid stability of liposomes. Optical density measurements at 400 nm were employed to probe the integrity of DSPC and ALA_{11,17}/DSPC liposome formulations as a function of pH. In these experiments nanospheres of varying compositions (0, 5, 10, 15, 25, and 50 mol % ALA_{11,17}/DSPC) were prepared in PBS. The liposomes were treated with aliquots of 1% HCl (v/v) and the pH and optical density at 400 nm were recorded immediately after each addition. The data were normalized to the original optical density at pH 7.4 and plotted against pH (FIG. 6). The pH profiles of all liposome formulations show a steady absorbance at higher pH values (pH>4.5). The pH profile of DSPC liposomes indicates sharply decreasing optical density below pH 3.5, consistent with phosphatidylcholine-based nanosphere disassembly at low pH (Lee et al., 2004; Arien et al., 1993; Zuidan and Crommelin, 1995). The 5 mol % ALA_{11,17}/DSPC liposomes exhibited steady optical density to pH 3.5 after which the absorbance decreased, but markedly less than that of the DSPC sample. In contrast, the 10 mol % ALA_{11,17}/DSPC liposome sample maintained a steady optical density over the entire pH range (7.4-1.9). The constancy in turbidity throughout the titration exhibited by this sample suggests nanosphere persistence throughout this pH range. The 15, 25, and 50 mol % ALA_{11,17}/DSPC liposome pH profiles, on the other hand, showed increasing turbidity below pH 4.0 with absorbance maxima at pH 3.8, 3.3, and 2.5 (pHmax), respectively. At even lower pH (<pHmax), the optical densities of each preparation decreased steadily, returning to the original turbidity near pH 2. These observations suggest that liposomes with ALA_{11,17} proportions greater than 10 mol % undergo aggregation at pH<4, which is correlated with ALA carboxylate protonation and resulting nanosphere surface neutralization. Further acidification leads to DSPC phosphate protonation, increasing positive charge at the liposome surface, resulting in disaggregation due to repulsive interactions. Thus, DSPC liposomes constituted with 10 mol % ALA_{11,17} stabilize liposomes to acidic challenge and at higher proportions of ALA_{11,17}, reversible, pH-dependent aggregation behavior is observed.

To examine the effect of acidity on particle size distribution, freshly prepared DSPC and 10 mol % ALA_{11,17}/DSPC liposome samples were exposed to strongly acidic conditions (pH 1.9) for a period of 15 minutes and the resultant suspensions were subjected to DLS analysis (FIGS. 4C and 4D). The DSPC liposomes exhibited a dramatically increased size distribution (FIG. 4C) while the 10 mol % ALA_{11,17}/DSPC liposomes exhibited similar size distribution (100-200 nm) and mean size (136 nm; FIG. 4D) comparable to that observed at pH 7.4 (FIG. 4B).

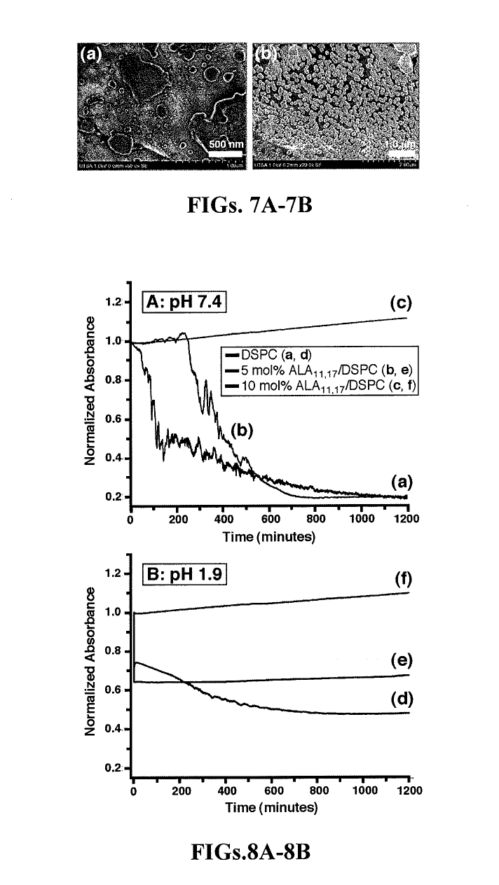

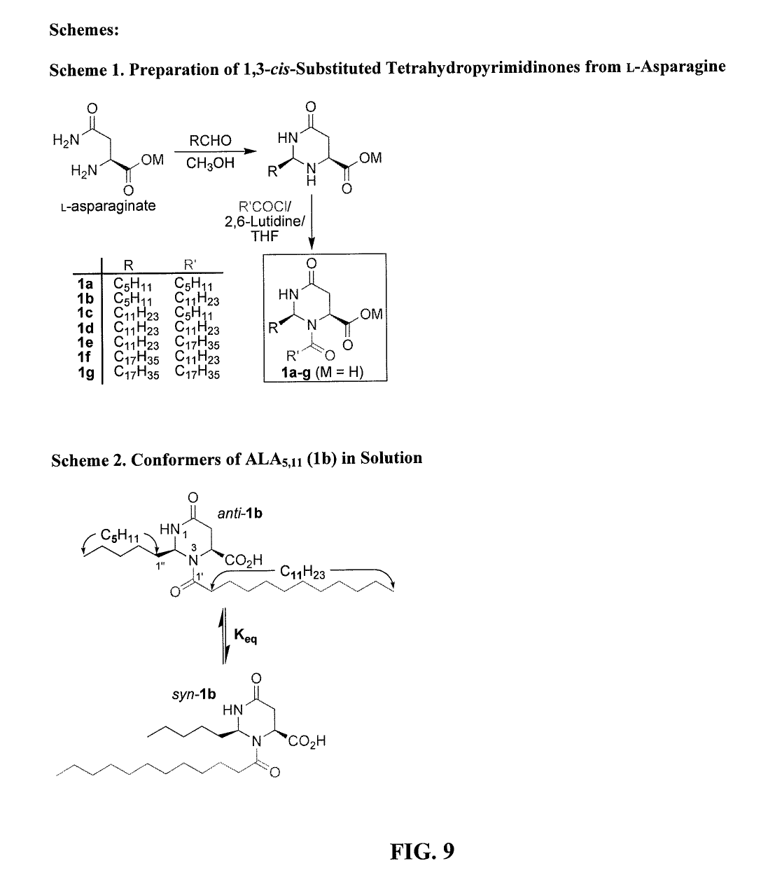

The morphologies of acidified samples of representative liposome formulations (DSPC and 10 mol % ALA_{11,17}/DSPC) were also examined by SEM (FIG. 7) to investigate nanosphere formation and aggregation behavior inferred from turbidity experiments (FIG. 6) and DLS analysis (FIG. 4). Thus, the DSPC and 10 mol % ALA_{11,17}/DSPC liposomes were freshly prepared at pH 7.4 and individual portions of each were acidified to pH 1.9. After standing at ambient conditions for 30 minutes, all four samples were examined by SEM (data was collected at pH in the vicinity of 7.4 and 1.9; FIGS. 5 and 7, respectively). The SEM image of DSPC nanospheres at pH 1.9 revealed non-spherical structures of various sizes (FIG. 7A), in contrast to the clustered, but unfused nanospheres that had been observed at pH 7.4 (FIG. 5A), suggesting their spontaneous degradation and reformulation under acidic conditions. SEM image of 10 mol % ALA_{11,17}/DSPC liposomes displayed intact nanospheres at pH as low as 1.9 (FIG. 7B) and exhibited the similar size distribution in acidic conditions as at pH 7.4 (FIG. 5B). These studies support the interpretation of the results of both the turbidity and DLS experiments that showed the ALA_{11,17}/DSPC liposomes (≧10 mol %) remain stable at pH<2.

The stabilities of selected liposomes to acidic environment were also examined as a function of time. Liposomes formed with DSPC, and 5 and 10 mol % ALA_{11,17}/DSPC were utilized to compare the persistence of nanosphere formulations under neutral and acidic conditions. In this experiment, the normalized optical densities of the liposome suspensions were monitored at 400 nm over a period of 20 hours (FIG. 8). Freshly prepared DSPC liposomes at pH 7.4 exhibited a rapid decrease in optical density over a 90 minute period during which precipitation occurred concurrently with mother liquor clarification (FIG. 8A), suggesting the liposome precipitation due to rapid aggregation and hence the loss of turbidity (vide supra). The liposomes containing 5 mol % ALA_{11,17} at pH 7.4 exhibited initial stability (>300 min; FIG. 8B), but the absorbance decreased rapidly after 4 h. In contrast, the turbidity of 10 mol % ALA_{11,17}/DSPC liposome sample remained relatively constant over the 20 h period. These results suggest that colloidal resistance to aggregation is improved with increasing proportions of novel lipid and that 10% ALA liposomes exhibit prolonged stability under neutral conditions (FIG. 8C).

Liposome persistence was similarly investigated under acidic conditions. Thus, freshly prepared DSPC and ALA_{11,17}/DSPC (5 and 10 mol %) liposome samples were acidified to pH 1.9 and the normalized absorbance was monitored as a function of time. The DSPC liposome sample exhibited an immediate and substantial loss of optical density, consistent with rapid liposome disassembly (FIG. 8D). The 5 mol % ALA_{11,17}/DSPC liposome sample also exhibited an abrupt loss of optical density, but maintained steady optical density thereafter (FIG. 8E). The 10 mol % ALA_{11,17}/DSPC liposome sample, on the other hand, maintained steady optical density at pH 1.9 (FIG. 8F) similar to that at pH 7.4. The results suggest that acidification of DSPC or 5 mol % ALA_{11,17}/DSPC liposomes to pH 1.9 causes immediate loss of nanosphere integrity as indicated by a rapid decline in optical density (Hayashi et al., 1995), while 10 mol % ALA_{11,17}/DSPC liposomes maintained colloidal stability regardless of the pH in ambient condition. Thus, these results support the interpretation that novel lipid ALA_{11,17} inhibits aggregation at neutral pH when used in 5 or 10 mol %, and stabilizes liposomes in acidic environments at 10 mol % formulations.

The DLS, SEM, and turbidity studies indicate that ALAs impart a dramatic stabilization to DSPC liposomes under both neutral and acidic conditions. Phosphatidylcholine nanosphere stabilization under neutral conditions is well known for preparations containing lipids with carboxylic acid head groups (Yu et al., 2007). The dramatic stabilities of these mixed lipid-containing liposomes to acidic conditions may also be associated with the intrinsically conical shape of the lipids. The pH dependent nanosphere aggregation-disaggregation phenomenon observed in these studies is correlated to liposome surface charge generated upon carboxylate and phosphate protonation. Additional studies are in progress to examine these issues.

TABLE 1
Novel Asparagine-Based Lipid Analogues
(ALA_{n, m}) and Their Respective Physical Data^{a}

| | | R | R′ | | | MW | | Mp. |
|---|---|---|---|---|---|---|---|---|
| Entry | Lipid^{b} | (CₙH₂ₙ₊₁) | (CₘH₂ₘ₊₁) | Yield^{c} | K_{eq}^{d} | (g/mol) | ([α]⁵⁸⁹)^{e} | (° C.) |
| 1 | 1a (ALA_{5, 5}) | C₅H₁₁ | C₅H₁₁ | 91 | 1.50 | 312.40 | −72.7 | 55-56 |
| 2 | 1b (ALA_{5, 11}) | C₅H₁₁ | C₁₁H₂₃ | 78 | 1.81 | 396.56 | −62.5 | 118-120 |
| 3 | 1c (ALA_{11, 5}) | C₁₁H₂₃ | C₅H₁₁ | 82 | 2.00 | 396.56 | −62.3 | 106-107 |
| 4 | 1d (ALA_{11, 11}) | C₁₁H₂₃ | C₁₁H₂₃ | 74 | 2.08 | 480.72 | −44.6 | 110-112 |
| 5 | 1e (ALA_{11, 17}) | C₁₁H₂₃ | C₁₇H₃₅ | 81 | 2.03 | 564.88 | −37.8 | 105-109 |
| 6 | 1f (ALA_{17, 11}) | C₁₇H₃₅ | C₁₁H₂₃ | 67 | 2.03 | 564.88 | −42.0 | 104-107 |
| 7 | 1g (ALA_{17, 17}) | C₁₇H₃₅ | C₁₇H₃₅ | 80 | 2.13 | 649.04 | N/A^{f} | 107-111 |

^{a}All compounds are in free acid form (1a-g; M = H) and their physical data were reported accordingly.
^{b}The corresponding ALA_{n, m} designations of the compounds are in parenthesis.
^{c}Net yield after two steps (Scheme 1).
^{d}K_{eq} ([syn − 1]/[anti − 1]) values were determined from the integrations of H-4 of ¹H NMR.
^{e}Optical rotations were measured in 9.0 mM solution of each free acid in methanol at 22° C.
^{f}ALA_{17, 17} gave turbid solutions in common solvents (DMSO, DMF, THF, DCM, and chloroform), preventing the determination of the specific rotation.

### REFERENCES

The following references, to the extent that they provide exemplary procedural or other details supplementary to those set forth herein, are specifically incorporated herein by reference.
U.S. Pat. No. 3,536,809
U.S. Pat. No. 3,598,123
U.S. Pat. No. 3,790,492
U.S. Pat. No. 3,845,770
U.S. Pat. No. 3,916,899
U.S. Pat. No. 4,008,719
U.S. Pat. No. 4,267,235
U.S. Pat. No. 4,326,008
U.S. Pat. No. 4,454,106
U.S. Pat. No. 4,472,509
U.S. Pat. No. 4,485,045
U.S. Pat. No. 4,487,855
U.S. Pat. No. 4,544,545
U.S. Pat. No. 4,613,559
U.S. Pat. No. 4,613,559
U.S. Pat. No. 4,774,189
U.S. Pat. No. 4,829,101
U.S. Pat. No. 4,831,175
U.S. Pat. No. 4,996,265
U.S. Pat. No. 5,013,556
U.S. Pat. No. 5,013,556
U.S. Pat. No. 5,073,498
U.S. Pat. No. 5,077,057
U.S. Pat. No. 5,100,591
U.S. Pat. No. 5,194,300
U.S. Pat. No. 5,266,497
U.S. Pat. No. 5,326,692
U.S. Pat. No. 5,395,619
U.S. Pat. No. 5,395,619
U.S. Pat. No. 5,395,688
U.S. Pat. No. 5,534,502
U.S. Pat. No. 5,573,909
U.S. Pat. No. 5,576,016
U.S. Pat. No. 5,616,334
U.S. Pat. No. 5,631,018
U.S. Pat. No. 5,644,048
U.S. Pat. No. 5,661,130
U.S. Pat. No. 5,723,218
U.S. Pat. No. 5,786,219
U.S. Pat. No. 5,952,131
U.S. Pat. No. 6,046,777
U.S. Pat. No. 6,071,533
U.S. Pat. No. 6,165,442
U.S. Pat. No. 6,177,062
U.S. Pat. No. 6,193,951
U.S. Pat. No. 6,219,572
U.S. Pat. No. 6,248,363
U.S. Pat. No. 6,287,857
U.S. Pat. No. 6,309,842
U.S. Pat. No. 6,406,713
U.S. Pat. No. 6,448,392
U.S. Pat. No. 6,720,001
U.S. Pat. No. 6,964,747
U.S. Pat. No. 7,439,250
Allen and Cullis, Science, 303:1818-1822, 2004.
Allen et al., Advanced Mat., 14:1562-+, 2002.
Allen et al., Inorg. Chem., 42:6300-6305, 2003.
Al-Meshal et al., Int. J. Pharmaceutics, 168:163-168, 1998.
Alving, In: Liposomes: From Biophysics to Therapeutics, Ostro (Ed.), 6:195-218, Marcel Dekker, Inc., NY, 1987.
Arién et al., Life Sci., 53:1279-1290, 1993.
Aucagne et al., Org. Lett., 8(20): 4505-7, 2006.
Beaucage et al., Tetrahedron, 49(10):1925, 1993.
Bird et al., Science, 242:423-426, 1988.
Bondi et al., Drug Delivery, 10:245-250, 2003.
Briu et al., J. Am. Chem. Soc., 111:2321, 1989.
Brower, Nature Biotechnol., 16:1304-1305, 1998.
Buttino et al., J. Exp. Biol., 209:801-809, 2006.
Camilleri et al., Am. J. Physiol.: Gastrointest. Liver Physiol., 257:G284-G290, 1989.
Carafa et al., Int. J. Pharm., 325:155-162, 2006.
Carlsson et al., Nature, 380:207, 1996.
Chonn and Cullis, Curr. Opin. Biotechnol., 6:698-708, 1995.
Chu et al., J. Am. Chem. Soc., 114:1800-1812, 1992.
Cohen et al., Proc. Natl. Acad. Sci. USA, 88:10440-10444, 1991.
Corbett and Gleason, Tetrahedron Letters, 43:1369-1372, 2002.
Crommelin and Schreier, In: Drugs and the Pharmaceutical Sciences Series, Kreuter (Ed.) 66(3):73-190, Marcel Dekker, Inc., NY, 1994.
DE 3,218,121
Deamer and Uster, In: Liposomes, Ostro (Ed.), Marcel Dekker, NY, 27-51, 1983.
De\check{g}im et al., J. Nanosci. Nanotech., 6:2945-2949, 2006.
Dong and Rogers, J. Microencapsulation, 9:437-448, 1992.
Dong and Rogers, Pharm. Res., 10:141-146, 1993.
Dougherty et al., J. Natl. Cancer Inst., 90(12):889-905, 1998.
Douglas et al., In: Hybrid Organic-inorganic Composites, Mark and Bianconi (Ed.), American Chem. Soc., Washington, D.C., 1995.
Douglas et al., Science, 269:54-57, 1995.
Douglas, In: Biomimetic Materials Chemistry, Mann (Ed.), VCH Publishers, NY, 1996.
Eckstein, In: Oligonucleotides and Analogues: A Practical Approach, Oxford University Press
Egholm, J. Am. Chem. Soc., 114:1895, 1992.
EP 102,324
EP 142,641
EP 143,949
EP 36,676
EP 52,322
EP 88,046
Epstein et al., Proc. Natl. Acad. Sci., USA, 82:3688, 1985.
Erickson et al., J. Org. Chem., 31:480-484, 1966.
Fingl et al., In: The Pharmacological Basis of Therapeutics, Ch. 1, 1975.
Flenniken et al., Chem. Commun., 447-449, 2005.
Flenniken et al., Nano Letters, 3:1573-1576, 2003.
Gabizon, In: Nanoparticulates as Drug Carriers, Torchilin (Ed.), 7:437-462, Imperial College Press, UK, 2006.
Gianolio and McLaughlin, Bioorg. Med. Chem., 9(9):2329-23234, 2001.
Gillies and Fréchet, Pure Appl. Chem., 76:1295-1307, 2004.
Gillitzer et al., Chem. Commun., 2390-2391, 2002.
Gregoriadis, In: Liposomes as Drug Carriers: Recent Trends and Progress, John Wiley and Sons Ltd., Chichester, UK, 1988.
Gregory et al., Infect. Immun., 54:780-786, 1986.
Gruner, In: Liposomes: from Biophysics to Therapeutics, Ostro (Ed.), 1:1-38, Marcel Dekker, NY, 1987.
Gular et al., Chem. Phys/Lipids, 160:33-44, 2009.
Gulati et al., Int. J. Pharm., 165:129-168, 1998.
Guyton and Hall, In: Textbook of Medical Physiology, 11^{th} Ed., Elsevier Saunders, PA, 2006.
Halter et al., Langmuir, 20:2416-2423, 2004.
Han et al., Lipids, 32:769-773, 1997.
Handjani-Vila et al., In: Liposome Technology, Gregoriadis (Ed.), 2(12):201-214, 2^{nd} Ed., CRC Press Inc., FL, 1993.
Hardy and Samworth, J. Chem. Soc. [Perkin 1](17):1954-1960, 1977.
Hartmuth et al., Angewandte Chemie-Internatl. Ed., 40(11):2004-21, 2001.
Hayashi et al., Chem. Pharm. Bull., 43:1751-1754, 1995.
Hayward and Smith, Cosmet. Toilet., 105:47-54, 1990.
Heath, Pediatr. Infect. Dis. J., 17:S117-S122, 1998.
Hermanson, Academic Press, San Diego, 1996.
Holliger et al., Proc. Natl. Acad. Sci. USA, 90:6444-6448, 1993.
Hui, Biophysical. J., 65:1362-1363, 1993.
Huston et al., Proc. Natl. Acad. Sci. USA, 85:5879-5883, 1988.
Hwang et al. (1980) Proc. Natl. Acad. Sci., USA, 77: 4030
Japanese Appln. 83-118008
Keller, Trends Food Sci. Technol., 12:25-31, 2001.
Kibat et al., FASEB J., 4:2533-2539, 1990.
Kirby et al., Biochem. J., 186:591-598, 1980.
Kirby, Food Sci. Technol. Today, 5:74-78, 1991.
Kirby, In: Controlled delivery of functional food ingredients: Opportunities for liposomes in the food industry, Liposome Technology, Gregoriadis (Ed.), 2(13):215-232, 2^{nd} Ed., CRC Press Inc., FL, 1993.
Kreimer-Bimbaum, Sem. Hematol., 26:157-73, 1989.
Kshirsagar et al., J. Postgrad. Med., 51:5-15, 2005.
Kubo et al., Arch. Biochem. Biophys., 410:141-148, 2003.
Kulkarni et al., J. Microencapsulation, 12:229-246, 1995.
Lakner and Negrete, Synlett, 643-645, 2002.
Langer, Science, 249:1527-1533, 1990.
Lasic and Barenholz, In: Handbook of Nonmedical Applications of Liposomes: From Gene Delivery and Diagnosis to Ecology, Vol. IV, CRC Press Inc., FL, 1996.
Lawson et al., Adv. Funct. Mater., 15:267-272, 2005.
Lawson et al., Langmuir, 19:6401-6407, 2003.
Lee et al., Drug Develop. Res., 61:13-18, 2004.
Lee et al., J. Am. Chem. Soc., 129:15096-15097, 2007.
Letsinger et al., J. Am. Chem. Soc., 110:4470, 1988.
Letsinger et al., Nucl. Acids Res., 14:3487, 1986.
Letsinger, J. Org. Chem., 35:3800, 1970.
López-Pinto et al., Int. J Pharm., 298:1-12, 2005.
MacLachlan, 2006, In: Antisense Drug Tech., Principles, Strategies and Applications, Crooke (Ed.), 2^{nd} Ed., 9:237-269, CRC Press Inc., Boca Raton, Fla.
Mag et al., Nucleic Acids Res., 19:1437, 1991.
Mansy, Int. J. Mol. Sci., 10:835-843, 2009.
McLean and Phillips, Biochemistry, 20:2893-2900, 1981.
Mecke et al., Langmuir, 19:2080-2087, 2003.
Meier et al., Chem. Int. Ed. Engl., 31:1008, 1992.
Michalek et al., In: Mucosal Immunology, Mestecky et al. (Eds.), 55:987-1008, 3^{rd} Ed., Elsevier Inc., Burlington, Mass., 2005.
Morigaki and Walde, Curr. Opin. Colloid Interface Sci., 12:75-80, 2007.
Müller et al., Langmuir, 21:7044-7051, 2005.
Murray, In: Colloidal synthesis of nanocrystals and nanocrystal superlattices, IBM J. Res. Dev., 45(1):47-56, 2001.
Nagumo et al., Chem. Pharm. Bull., 39:1889-1891, 1991.
Ng et al., Macromolecules, 34:5759-5765, 2001.
Nielsen, Nature, 365:566, 1993.
Osanai and Nakamura, Biomaterials, 21:867-876, 2000.
Papahadjopoulos et al., Proc. Natl. Acad. Sci. USA, 88:11460-11464, 1991.
Park, J. Breast Cancer Res., 4:95-99, 2002.
Pauwels et al., Chemica Scripta, 26:141, 1986.
PCT Appln. PCT/US92/09965
PCT Appln. WO 97/14443
PCT Appln. WO94/13804
Pedersen et al., Eur. J. Pharm. Biopharm., 62:155-162, 2006.
Phillips and Goins, In: Handbook of Targeted Delivery of Imaging Agents, Torchilin (Ed.), 10:149-173, CRC Press, Inc., FL, 1995.
Picón et al., Biotechnol. Lett., 19:345-348, 1997.
Regen, In: Liposomes: From Biophysics to Therapeutics, Ostro (Ed.), 3:73-108, Marcel Dekker, Inc., NY, 1987.
Remington's Pharmaceutical Sciences, 17^{th} Ed., Mack Publishing Company, PA, 1985.
Rivest et al., Pharm. Res., 24:981-990, 2007.
Rosenzqeig et al., Bioconjugate Chem., 11:306-313, 2000.
Sakaguchi et al., Bioconjugate Chem., 19:1040-1048, 2008.
Sawai et al, Chem. Lett., 805, 1984.
Schiffelers et al., Nucleic Acids Res., 32:e149, 2004.
\check{S}entjurc et al., J. Controlled Rel., 59:87-97, 1999.
Sharma and Sharma, Int. J. Pharm., 154:123-140, 1997.
Shiokawa et al., Clin. Cancer Res., 11:2018-2025, 2005.
Siegal et al., J. Neurosurgery, 83:1029-1037, 1995.
Sou et al., Biomaterials, 28:2655-2666, 2007.
Sprinzl et al., Eur. J. Biochem., 81:579, 1977.
Stanzi In: Cosmetic Science and Technology Series, Magdassi and Touitou (Eds.), 19(12):233-265, 2^{nd} Ed., Marcel Dekker, Inc., NY, 1999.
Strauss, J. Soc. Cosmet. Chem., 40:51-60, 1989.
Subczynski et al., Biochim. Biophys. Acta, 1150:173-181, 1993.
Szoka and Papahadjopoulos, Ann. Rev. Biophys. Bioeng., 9:467, 1980.
Tanford, In: The Hydrophobic Effect: Formation of Micelles and Biological Membranes, 2^{nd} Ed., John Wiley and Sons, Inc. NY.
Tomlinson et al., Methods Enzymol., 326:461-479, 2000.
Torchilin, Nature Rev. Drug Discovery, 4:145-160, 2005.
Torrado et al., J. Pharm. Sci., 97:2405-2425, 2008.
Valenti et al., J. Liposome Res., 11:73-90, 2001.
Vikbjerg et al., J. Am. Oil Chem. Soc., 84:631-637, 2007.
Wang et al., Angewandte Chemie-Internatl. Ed., 41:459-462, 2002c.
Wang et al., Chem. Biol., 9:805-811, 2002a.
Wang et al., Chem. Biol., 9:813-819, 2002b.
Wang, In: Drug Delivery: Principles and Applications, Wang et al. (Eds.), 19:411-434, John Wiley and Sons, Inc., NJ, 2005.
Ward et al., Nature, 341:544-546, 1989.
Werle and Takeuchi, Int. J. Pharm., 370:26-32, 2009.
Xia and Xu, Food Res. Int., 38:289-296, 2005.
Yu et al., J. Phys. Chem. C, 111:8233-8236, 2007.
Zalipsky et al., In: Stealth Liposomes, Pharmacology and Toxicology, 233:244, CRC Press, FL, 1995.
Zuidam and Crommelin, J. Pharm. Sci., 84:1113-1119, 1995.

What is claimed is:

1. A method of administering a therapeutic or diagnostic agent to the intestines of a subject comprising orally administering a lipidic particle comprising at least 5, 10, 15, 20, 40, 50, or 60 mol % ALA, CLA, or SLA, and a therapeutic or diagnostic agent to a subject.

2. The method of claim 1, wherein the lipidic particle further comprises 40, 50, 60, 70, 80, 90, or 95 mol % of a phospholipid.

3. The method of claim 2, wherein the phospholipid is phosphatidylcholine.

4. The method of claim 3, wherein the phosphatidylcholine is distearoylphosphatidylcholine (DSPC).

5. The method of claim 1, wherein the lipidic particle further comprises 0.01% to 20 mol % of a stabilizing agent.

6. The method of claim 5 wherein the stabilizing agent is cholesterol, cholesterol esters, cholestanol, glucoronic acid derivatives, polysaccharide, saturated fatty acids, unsaturated fatty acids, and/or polyethylene glycol.

7. The method of claim 1, wherein the lipidic particle is a liposome.

8. The method of claim 1, wherein the therapeutic or diagnostic agent is an antigen, an antibiotic, a peptide, a pharmaceutical, a nucleic acid, a detectable agent, and/or an antibody.

9. The method of claim 8, wherein the detectable agent is a radiographic contrast agent.

10. A method of delivering an antigen to the small intestines comprising orally administering an antigen encapsulated in a lipidic particle comprising at least 5, 10, 15, 20, 40, or 60 mol % ALA, CLA, or SLA, and an encapsulated acid labile agent to a subject.

11. An acid stable lipid particle composition comprising:
(a) at least 5, 10, 15, 20, 40, or 60 mol % of a lipopeptide derivative selected from an asparagine, cysteine, or serine lipid derivative having the formula ALA_{R1,R2}, CLA_{R1,R2}, or SLA_{R1,R2} wherein in R1 and R2 are independently an alkyl chain of at least 10, 11, 12, 13, 14, 15, 16, 17, 18, 19, or 20 carbons;
(b) at least 40, 60, 80, 90, 95 mol % amphiphilic lipid; and
(c) a therapeutic or diagnostic agent;
wherein the lipid particle is stable for at least 10 hours at a pH of 0.5 to 4 at room temperature.

12. The particle of claim 11, where in R1 is 11 and R2 is 17.

13. The particle of claim 11, wherein the lipidic particles are 20 μm to 200 μm in diameter.

14. The particle of claim 11, further comprising a targeting moiety.

15. The particle of claim 11, further comprising a detectable label.

16. A method of modulating lipidic particle size by adjusting the ALA content, wherein an increase in ALA results in a decrease in lipidic particle size.

17. A method of modulating acid stability of a lipid particle by adjusting the length of the alkyl chains of a ALA, CLA, or SLA component of the lipid particle, wherein the shorter the alkyl chain the less stable the lipid particle.

18. A method of preparing lipid particle having a size range of 20 μm to 200 μm without extruding the lipidic mixture comprising:
(a) combining an ALA, CLA, or SLA, and an amphiphilic lipid, wherein the ALA, CLA, or SLA component is 5, 10, 20, 40, or 60 mol % of the combination and the amphiphilic component is 40, 50, 60, 70, 80, 90, 95 mol % of the combination;
(b) preparing a thin film of the combination;
(c) hydrating the thin film forming self-assembled lipid particles comprising at least 5 mol % ALA, CLA, or SLA.
